(19) United States
(12) Patent Application Publication
(10) Pub. No.: US 2021/0316006 A1
(43) Pub. Date: Oct. 14, 2021

(54) PYRROLOBENZODIAZEPINE CONJUGATES

(71) Applicant: MEDIMMUNE LIMITED, Cambridge (GB)

(72) Inventors: Philip Wilson HOWARD, Cambridge (GB); Ian HUTCHINSON, Cambridge (GB)

(21) Appl. No.: 17/285,811

(22) PCT Filed: Oct. 18, 2019
(86) PCT No.: PCT/EP2019/078402

§ 371 Date: Apr. 15, 2021

(30) Foreign Application Priority Data
Oct. 19, 2018 (GB) 1817088.6
Jun. 7, 2019 (GB) 1908126.4

(51) Int. Cl. A61K 47/68; C07K 5/078; C07K 5/065; C07K 5/062

(52) U.S. Cl. CPC A61K 47/6855; C07K 5/06156; C07K 5/06078; C07K 5/06034; C07K 5/06043; C07K 5/06026; C07K 5/06052

(57) ABSTRACT

A compound of formula (I) wherein R^{L} is a linker for connection to a cell binding agent.

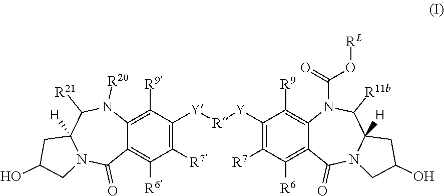

The present invention relates to conjugates comprising pyrrolobenzodiazepines and related dimers (PBDs), and the precursor drug linkers used to make such conjugates.

### BACKGROUND TO THE INVENTION

Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994)). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

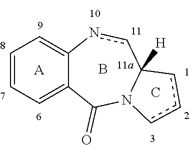

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe))ram at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

It has been previously disclosed that the biological activity of these molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity.

The first dimers (Bose, D. S., et al., J. Am. Chem. Soc., 114, 4939-4941 (1992)) were of the general formula:

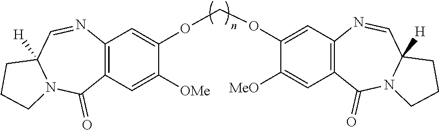

where n is from 3 to 6. The compounds where n were 3 and 5 showed promising cytoxicity in vitro. However when antitumor activity of the the n=3 compound (DSB-120) was studied (Walton, M., et al., Cancer Chemother Pharmacol (1996) 38: 431. doi:10.1007/s002800050507), this was found not to be as promising. This lack of promise was thought to be “a consequence of low tumour selectivity and drug uptake as a result of high protein binding and/or extensive drug metabolism in vivo”.

In order to improve on these compounds, compounds were investigated (Gregson, S. J., et al., Chem. Commun., 1999, 797-798. doi: 10.1039/A809791G) with the “inclusion of C2/C2′ substituents that should follow the contour of the host minor groove”. This compound SG2000 (SJG-136):

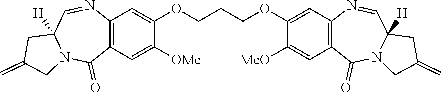

was found to have “exquisite cytotoxicity in the picomolar region . . . some 9000-fold more potent that DSB-120.”

This compound (also discussed in Gregson, S., et al., J. Med. Chem., 44, 737-748 (2001); Alley, M. C., et al., Cancer Research, 64, 6700-6706 (2004); and Hartley, J. A., et al., Cancer Research, 64, 6693-6699 (2004)) has been involved in clinical trials as a standalone agent, for example, NCT02034227 investigating its use in treating Acute Myeloid Leukemia and Chronic Lymphocytic Leukemia (see: https://www.clinicaltrials.gov/ct2/show/NCT02034227).

Dimeric PBD compounds bearing C2 aryl substituents alongside endo-unsaturation, such as SG2202 (ZC-207), are disclosed in WO 2005/085251:

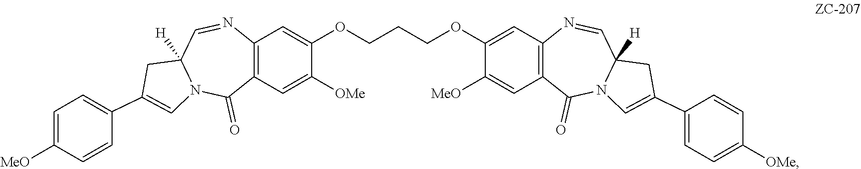 [SMILES: COc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

and in WO2006/111759, bisulphites of such PBD compounds, for example SG2285 (ZC-423):

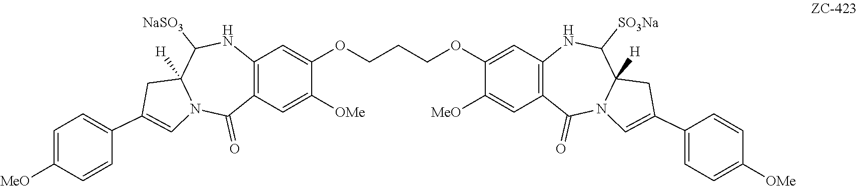

These compounds have been shown to be highly useful cytotoxic agents (Howard, P. W., et al., Bioorg. Med. Chem. (2009), doi: 10.1016/j.bmcl.2009.09.012).

In a review of PBD containing ADCs (Mantaj, J., et al., Angew. Chem. Int. Ed. (2016), 55, 2-29; DOI: 10.1002/anie.201510610), the SAR of PBD dimers is discussed. The summary of the SAR is presented in FIG. 3—B “C2-exo and C1-C2/C2-C3 unsaturation enhances activity”. A more detailed discussion is found at section 2.4 which says:

“DSB-120 has poor activity in vivo, attributed partly to its high reactivity with cellular thiol-containing molecules such as glutathione. However, introduction of C2/C2′-exo unsaturation as in SJG-136 led to an overall increase in DNA-binding affinity and cytotoxicity, and a lower reactivity toward cellular nucleophiles with more of the agent potentially reaching its target DNA.”

WO 2007/085930 describes the preparation of dimer PBD compounds having linker groups for connection to a cell binding agent, such as an antibody. The linker is present in the bridge linking the monomer PBD units of the dimer.

Dimer PBD compounds having linker groups for connection to a cell binding agent, such as an antibody, are described in WO 2011/130598. The linker in these compounds is attached to one of the available N10 positions, and are generally cleaved by action of an enzyme on the linker group. The dimer PBD compounds have either endo or exo unsaturation in the C-ring.

WO 2014/057074 and WO 2015/052322 describes specific PBD dimer conjugates bound via the N10 position on one monomer, and all these compounds have endo unsaturation in the C-ring.

WO2014/096365 discloses the compound:

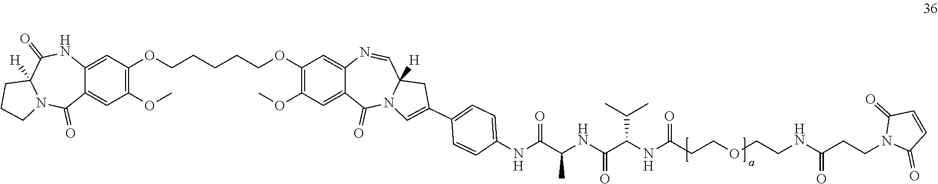

where the lack of unsaturation in the C-ring is coupled with the B-ring being a dilactam and therefore not having the ability to covalently bind DNA.

### DISCLOSURE OF THE INVENTION

The present invention provides PBD dimer drug linkers and conjugates where neither C-ring has endo- or exo-unsaturation, and where both C2 positions bear a hydroxy group. A first aspect of the present invention comprises a compound with the formula I:

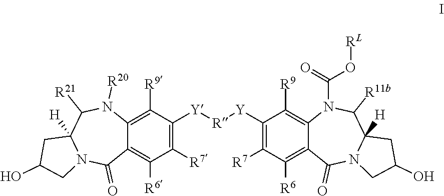

and salts and solvates thereof, wherein:
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
R^{11b} is selected from OH, OR^{A}, where R^{A} is C₁₋₄ alkyl; and
R^{L} is a linker for connection to a cell binding agent, which is selected from:
(iiia):

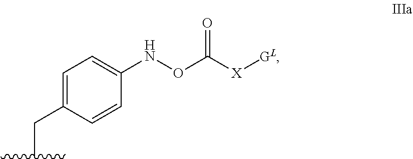

wherein

### Q is:

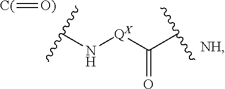

where Q^{X} is such that Q is an amino-acid residue, a dipeptide residue or a tripeptide residue;

### X is:

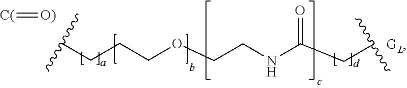

where a=0 to 5, b=0 to 16, c=0 or 1, d=0 to 5;
G^{L} is a linker for connecting to a Ligand Unit; and
(iiib):

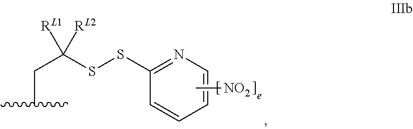

where R^{L1} and R^{L2} are independently selected from H and methyl, or together with the carbon atom to which they are bound form a cyclopropylene or cyclobutylene group;
and e is 0 or 1;
either:
(a) R²⁰ is H and R²¹ is H;
(b) R²⁰ is H and R²¹ is ═O; or
(c) R²¹ is OH or OR^{A}, where R^{A} is C₁₋₄ alkyl and R²⁰ is selected from:

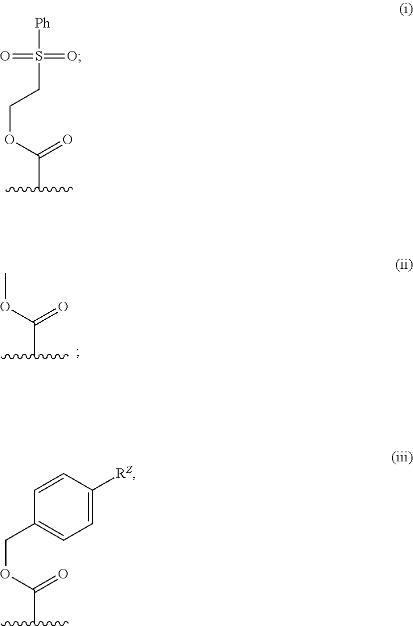

where R^{Z} is selected from:

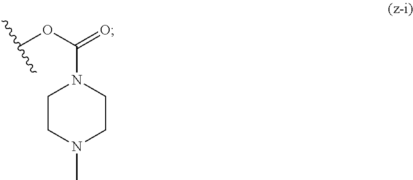 [SMILES: *OC(=O)N1CCN(C)CC1]

(z-ii) OC(═O)CH₃;
(z-iii) NO₂;
(z-iv) OMe;
(z-v) glucoronide;
(z-vi) NH—C(═O)—X₁—NHC(═O)X₂—NH—C(═O)—R^{ZC}, where —C(═O)—X₁—NH— and —C(═O)—X₂—NH— represent natural amino acid residues and R^{ZC} is selected from Me, OMe, CH₂CH₂OMe, and (CH₂CH₂O)₂Me.

In alternative embodiments, R⁷ and R^{7′} may together form a group which is: (i) —O—(CH₂)ₙ—O—, where n is from 7 to 16; or (ii) —O—(CH₂CH₂O)ₘ—, where m is 2 to 5.

Such drug linkers have been found to undergo ready conjugation to ligand units such as antibodies. The presence of the R²⁰ group is believed to be important to avoid cross-reaction between the C2-OH groups and the N10-C11 imine group. Equally, replacing the N10-C11 with a secondary amine or lactam group avoids this issue.

A second aspect of the present invention provides Conjugates of formula II:

L-(D^{L})ₚ (II)

wherein L is a Ligand unit (i.e., a targeting agent), D^{L} is a Drug Linker unit of formula I′:

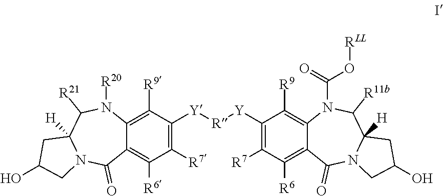

wherein R⁶, R⁷, R⁹, R^{11b}, Y, R″, Y′, R^{6′}, R^{7′}, R^{9′}, R²⁰ and R²¹ are as defined in the first aspect of the invention;
R^{LL} is a linker for connection to a cell binding agent, which is selected from:
(iiia):

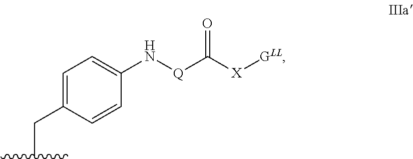

where Q and X are as defined in the first aspect and G^{LL} is a linker connected to a Ligand Unit; and
(iiib):

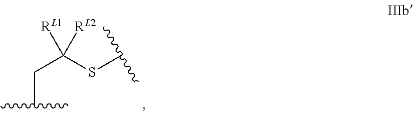

where R^{L1} and R^{L2} are as defined in the first aspect;
wherein p is an integer of from 1 to 20.

The Ligand unit, described more fully below, is a targeting agent that binds to a target moiety. The Ligand unit can, for example, specifically bind to a cell component (a Cell Binding Agent) or to other target molecules of interest. The Ligand unit can be, for example, a protein, polypeptide or peptide, such as an antibody, an antigen-binding fragment of an antibody, or other binding agent, such as an Fc fusion protein.

These conjugates have been found to possess a high tolerability which leads to a high therapeutic index, thus making them promising candidates for clinical development.

A third aspect of the present invention provides the use of a conjugate of the second aspect of the invention in the manufacture of a medicament for treating a proliferative disease. The third aspect also provides a conjugate of the second aspect of the invention for use in the treatment of a proliferative disease. The third aspect also provides a method of treating a proliferative disease comprising administering a therapeutically effective amount of a conjugate of the second aspect of the invention to a patient in need thereof.

One of ordinary skill in the art is readily able to determine whether or not a candidate conjugate treats a proliferative condition for any particular cell type. For example, assays which may conveniently be used to assess the activity offered by a particular compound are described in the examples below.

A fourth aspect of the present invention provides the synthesis of a conjugate of the second aspect of the invention comprising conjugating a compound (drug linker) of the first aspect of the invention with a Ligand Unit.

A fifth aspect of the present invention provides a compound of formula IV:

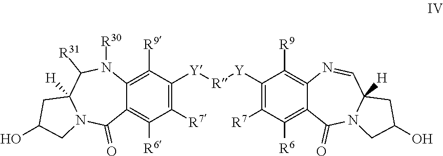

wherein R⁶, R⁷, R⁹, Y, R″, Y′, R^{6′}, R^{7′} and R^{9′} are as defined in the first aspect of the invention;
either:
(a) R³⁹ is H and R³¹ is H;
(b) R³⁹ is H and R³¹ is ═O; or
(c) R³⁹ and R³¹ form a double bond between the N and C atoms to which they are attached.

Compounds of formula IV are the warheads released by conjugates of the first aspect.

### Definitions

Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:
   norcarane (C₇), norpinane (C₇), norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. The term “C₅₋₇ aryl”, as used herein, pertains to a monovalent moiety obtained by removing a hydrogen atom from an aromatic ring atom of an aromatic compound, which moiety has from 5 to 7 ring atoms and the term “C₅₋₁₀ aryl”, as used herein, pertains to a monovalent moiety obtained by removing a hydrogen atom from an aromatic ring atom of an aromatic compound, which moiety has from 5 to 10 ring atoms. Preferably, each ring has from 5 to 7 ring atoms.

In this context, the prefixes (e.g. C₃₋₂₀, C₅₋₇, C₅₋₆, C₅₋₁₀, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (Ow), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

### Halo: —F, —Cl, —Br, and —I.

### Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —O(═O)CH₃ (acetyl), —C(═O)CH₂CH₃ (propionyl), —O(═O)O(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

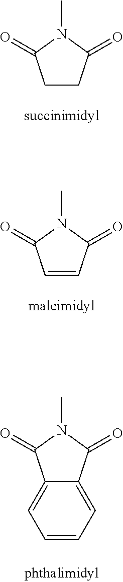

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

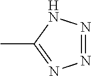

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group.

Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

Where the C₃₋₁₂ alkylene group is interrupted by a heteroatom, the subscript refers to the number of atoms in the chain including the heteroatoms. For example, the chain —C₂H₄—O—C₂H₄— would be a C₅ group.

Where the C₃₋₁₂ alkylene group is interrupted by a heteroatom, the subscript refers to the number of atoms directly in the chain including the aromatic ring. For example, the chain

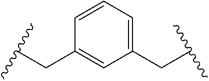 [SMILES: *Cc1cccc(C*)c1]

would be a C₅ group.

The symbols * and [symbol] are used interchangably to represent the attachment point of the chemical group.

Ligand Unit

The Ligand Unit may be of any kind, and include a protein, polypeptide, peptide and a non-peptidic agent that specifically binds to a target molecule. In some embodiments, the Ligand unit may be a protein, polypeptide or peptide. In some embodiments, the Ligand unit may be a cyclic polypeptide. These Ligand units can include antibodies or a fragment of an antibody that contains at least one target molecule-binding site, lymphokines, hormones, growth factors, or any other cell binding molecule or substance that can specifically bind to a target.

The terms “specifically binds” and “specific binding” refer to the binding of an antibody or other protein, polypeptide or peptide to a predetermined molecule (e.g., an antigen). Typically, the antibody or other molecule binds with an affinity of at least about 1×10⁷ M⁻¹, and binds to the predetermined molecule with an affinity that is at least two-fold greater than its affinity for binding to a non-specific molecule (e.g., BSA, casein) other than the predetermined molecule or a closely-related molecule.

Examples of Ligand units include those agents described for use in WO 2007/085930, which is incorporated herein.

In some embodiments, the Ligand unit is a Cell Binding Agent that binds to an extracellular target on a cell. Such a Cell Binding Agent can be a protein, polypeptide, peptide or a non-peptidic agent. In some embodiments, the Cell Binding Agent may be a protein, polypeptide or peptide. In some embodiments, the Cell Binding Agent may be a cyclic polypeptide. The Cell Binding Agent also may be antibody or an antigen-binding fragment of an antibody. Thus, in one embodiment, the present invention provides an antibody-drug conjugate (ADC).

Cell Binding Agent

A cell binding agent may be of any kind, and include peptides and non-peptides. These can include antibodies or a fragment of an antibody that contains at least one binding site, lymphokines, hormones, hormone mimetics, vitamins, growth factors, nutrient-transport molecules, or any other cell binding molecule or substance.

Peptides

In one embodiment, the cell binding agent is a linear or cyclic peptide comprising 4-30, preferably 6-20, contiguous amino acid residues. In this embodiment, it is preferred that one cell binding agent is linked to one monomer or dimer pyrrolobenzodiazepine compound.

In one embodiment the cell binding agent comprises a peptide that binds integrin αᵥβ₆. The peptide may be selective for αᵥβ₆ over XYS.

In one embodiment the cell binding agent comprises the A20FMDV-Cys polypeptide. The A20FMDV-Cys has the sequence: NAVPNLRGDLQVLAQKVARTC. Alternatively, a variant of the A20FMDV-Cys sequence may be used wherein one, two, three, four, five, six, seven, eight, nine or ten amino acid residues are substituted with another amino acid residue. Furthermore, the polypeptide may have the sequence NAVXXXXXXXXXXXXXXXRTC.

Antibodies

The term “antibody” herein is used in the broadest sense and specifically covers monoclonal antibodies, polyclonal antibodies, dimers, multimers, multispecific antibodies (e.g., bispecific antibodies), multivalent antibodies and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include chimeric antibodies, humanized antibodies and human antibodies.

Examples of cell binding agents include those agents described for use in WO 2007/085930, which is incorporated herein.

Tumour-associate antigens and cognate antibodies for use in embodiments of the present invention are listed below, and are described in more detail on pages 14 to 86 of WO 2017/186894, which is incorporated herein.

(1) BMPR1B (bone morphogenetic protein receptor-type IB)

### (2) E16 (LAT1, SLC7A5)

(3) STEAP1 (six transmembrane epithelial antigen of prostate)

### (4) 0772P (CA125, MUC16)

(5) MPF (MPF, MSLN, SMR, megakaryocyte potentiating factor, mesothelin)
(6) Napi3b (NAPI-3B, NPTIIb, SLC34A2, solute carrier family 34 (sodium phosphate), member 2, type II sodium-dependent phosphate transporter 3b)
(7) Sema 5b (FLJ10372, KIAA1445, Mm.42015, SEMA5B, SEMAG, Semaphorin 5b Hlog, 25 sema domain, seven thrombospondin repeats (type 1 and type 1-like), transmembrane domain (TM) and short cytoplasmic domain, (semaphorin) 5B)
(8) PSCA hlg (2700050C12Rik, C530008O16Rik, RIKEN cDNA 2700050C12, RIKEN cDNA
2700050012 gene)
(9) ETBR (Endothelin type B receptor)
(10) MSG783 (RNF124, hypothetical protein FLJ20315)
(11) STEAP2 (HGNC_8639, IPCA-1, PCANAP1, STAMP1, STEAP2, STMP, prostate cancer
associated gene 1, prostate cancer associated protein 1, six transmembrane epithelial antigen of prostate 2, six transmembrane prostate protein)
(12) TrpM4 (BR22450, FLJ20041, TRPM4, TRPM4B, transient receptor potential cation 5 channel, subfamily M, member 4)
(13) CRIPTO (CR, CR1, CRGF, CRIPTO, TDGF1, teratocarcinoma-derived growth factor)
(14) CD21 (CR2 (Complement receptor 2) or C3DR (C3d/Epstein Barr virus receptor) or Hs.73792)
(15) CD79b (CD79B, CD79β, IGb (immunoglobulin-associated beta), B29)
(16) FcRH2 (IFGP4, IRTA4, SPAP1A (SH2 domain containing phosphatase anchor protein 1a), SPAP1B, SPAP1C)

### (17) HER2 (ErbB2)

### (18) NCA (CEACAM6)

### (19) MDP (DPEP1)

### (20) IL20R-alpha (IL20Ra, ZCYTOR7)

### (21) Brevican (BCAN, BEHAB)

### (22) EphB2R (DRT, ERK, Hek5, EPHT3, Tyro5)

### (23) ASLG659 (B7h)

(24) PSCA (Prostate stem cell antigen precursor)

### (25) GEDA

(26) BAFF-R (B cell-activating factor receptor, BLyS receptor 3, BR3)
(27) CD22 (B-cell receptor CD22-B isoform, BL-CAM, Lyb-8, Lyb8, SIGLEC-2, FLJ22814)
(27a) CD22 (CD22 molecule)
(28) CD79a (CD79A, CD79alpha), immunoglobulin-associated alpha, a B cell-specific protein that covalently interacts with Ig beta (CD79B) and forms a complex on the surface with Ig M molecules, transduces a signal involved in B-cell differentiation), pl: 4.84, MW: 25028 TM: 2 [P] Gene Chromosome: 19q13.2).
(29) CXCR5 (Burkitt's lymphoma receptor 1, a G protein-coupled receptor that is activated by the CXCL13 chemokine, functions in lymphocyte migration and humoral defense, plays a
10 role in HIV-2 infection and perhaps development of AIDS, lymphoma, myeloma, and leukemia); 372 aa, pl: 8.54 MW: 41959 TM: 7 [P] Gene Chromosome: 11q23.3,
(30) HLA-DOB (Beta subunit of MHC class II molecule (la antigen) that binds peptides and 20 presents them to CD4+ T lymphocytes); 273 aa, pl: 6.56, MW: 30820.TM: 1 [P] Gene Chromosome: 6p21.3)
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).
(32) CD72 (B-cell differentiation antigen CD72, Lyb-2); 359 aa, pl: 8.66, MW: 40225, TM: 1 5 [P] Gene Chromosome: 9p13.3).
(33) LY64 (Lymphocyte antigen 64 (RP105), type I membrane protein of the leucine rich repeat (LRR) family, regulates B-cell activation and apoptosis, loss of function is associated
with increased disease activity in patients with systemic lupus erythematosis); 661 aa, pl: 6.20, MW: 74147 TM: 1 [P] Gene Chromosome: 5q12).
(34) FcRH1 (Fc receptor-like protein 1, a putative receptor for the immunoglobulin Fc domain
that contains C2 type Ig-like and ITAM domains, may have a role in B-lymphocyte 20 differentiation); 429 aa, pl: 5.28, MW: 46925 TM: 1 [P] Gene Chromosome: 1q21-1q22)
(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pl: 6.88, MW: 106468, TM: 1 [P] Gene Chromosome: 1q21)
(36) TENB2 (TMEFF2, tomoregulin, TPEF, HPP1, TR, putative transmembrane 35 proteoglycan, related to the EGF/heregulin family of growth factors and follistatin); 374 aa)
(37) PSMA—FOLH1 (Folate hydrolase (prostate-specific membrane antigen) 1)
(38) SST (Somatostatin Receptor; note that there are5 subtypes)
(38.1) SSTR2 (Somatostatin receptor 2)
(38.2) SSTR5 (Somatostatin receptor 5)

### (38.3) SSTR1

### (38.4) SSTR3

### (38.5) SSTR4

### AvB6—Both Subunits (39+40)

(39) ITGAV (Integrin, alpha V)
(40) ITGB6 (Integrin, beta 6)
(41) CEACAM5 (Carcinoembryonic antigen-related cell adhesion molecule 5)
(42) MET (met proto-oncogene; hepatocyte growth factor receptor)
(43) MUC1 (Mucin 1, cell surface associated)
(44) CA9 (Carbonic anhydrase IX)
(45) EGFRvIII (Epidermal growth factor receptor (EGFR), transcript variant 3,
(46) CD33 (CD33 molecule)
(47) CD19 (CD19 molecule)
(48) IL2RA (Interleukin 2 receptor, alpha); NCBI Reference Sequence: NM_000417.2);
(49) AXL (AXL receptor tyrosine kinase)
(50) CD30-TNFRSF8 (Tumor necrosis factor receptor superfamily, member 8)
(51) BCMA (B-cell maturation antigen)—TNFRSF17 (Tumor necrosis factor receptor superfamily, member 17)

### (52) CT Ags—CTA (Cancer Testis Antigens)

(53) CD174 (Lewis Y)—FUT3 (fucosyltransferase 3 (galactoside 3(4)-L-fucosyltransferase, Lewis blood group)
(54) CLEC14A (C-type lectin domain family 14, member A; Genbank accession no. NM175060)
(55) GRP78—HSPA5 (heat shock 70 kDa protein 5 (glucose-regulated protein, 78 kDa)
(56) CD70 (CD70 molecule) L08096
(57) Stem Cell specific antigens. For example:
5T4 (see entry (63) below)
CD25 (see entry (48) above)
CD32
LGR5/GPR49
Prominin/CD133

### (58) ASG-5

(59) ENPP3 (Ectonucleotide pyrophosphatase/phosphodiesterase 3)
(60) PRR4 (Proline rich 4 (lacrimal))
(61) GCC-GUCY2C (guanylate cyclase 2C (heat stable enterotoxin receptor)
(62) Liv-1-SLC39A6 (Solute carrier family 39 (zinc transporter), member 6)
(63) 5T4, Trophoblast glycoprotein, TPBG-TPBG (trophoblast glycoprotein)
(64) CD56-NCMA1 (Neural cell adhesion molecule 1)
(65) CanAg (Tumor associated antigen CA242)

### (66) FOLR1 (Folate Receptor 1)

(67) GPNMB (Glycoprotein (transmembrane) nmb)
(68) TIM-1-HAVCR1 (Hepatitis A virus cellular receptor 1)
(69) RG-1/Prostate tumor target Mindin-Mindin/RG-1
(70) B7-H4-VTCN1 (V-set domain containing T cell activation inhibitor 1
(71) PTK7 (PTK7 protein tyrosine kinase 7)
(72) CD37 (CD37 molecule)
(73) CD138-SDC1 (syndecan 1)
(74) CD74 (CD74 molecule, major histocompatibility complex, class II invariant chain)

### (75) Claudins-CLs (Claudins)

(76) EGFR (Epidermal growth factor receptor)
(77) Her3 (ErbB3)-ERBB3 (v-erb-b2 erythroblastic leukemia viral oncogene homolog 3 (avian))
(78) RON-MST1R (macrophage stimulating 1 receptor (c-met-related tyrosine kinase))
(79) EPHA2 (EPH receptor A2)
(80) CD20-MS4A1 (membrane-spanning 4-domains, subfamily A, member 1)

### (81) Tenascin C-TNC (Tenascin C)

(82) FAP (Fibroblast activation protein, alpha)
(83) DKK-1 (Dickkopf 1 homolog (Xenopus laevis)
(84) CD52 (CD52 molecule)
(85) CS1-SLAMF7 (SLAM family member 7)

### (86) Endoglin-ENG (Endoglin)

### (87) Annexin A1-ANXA1 (Annexin A1)

(88) V-CAM (CD106)-VCAM1 (Vascular cell adhesion molecule 1)

An additional tumour-associate antigen and cognate antibodies of interest are:

(89) ASCT2 (ASC transporter 2, also known as SLC1A5).
ASCT2 antibodies are described in WO 2018/089393, which is incorporated herein by reference.

The cell binding agent may be labelled, for example to aid detection or purification of the agent either prior to incorporation as a conjugate, or as part of the conjugate. The label may be a biotin label. In another embodiment, the cell binding agent may be labelled with a radioisotope.

Methods of Treatment

The compounds of the present invention may be used in a method of therapy. Also provided is a method of treatment, comprising administering to a subject in need of treatment a therapeutically-effective amount of a conjugate of formula II. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy.

Pharmaceutical compositions according to the present invention, and for use in accordance with the present invention, may comprise, in addition to the active ingredient, i.e. a conjugate of formula II, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The Conjugates can be used to treat proliferative disease and autoimmune disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g., histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin.

Examples of autoimmune disease include the following: rheumatoid arthritis, autoimmune demyelinative diseases (e.g., multiple sclerosis, allergic encephalomyelitis), psoriatic arthritis, endocrine ophthalmopathy, uveoretinitis, systemic lupus erythematosus, myasthenia gravis, Graves' disease, glomerulonephritis, autoimmune hepatological disorder, inflammatory bowel disease (e.g., Crohn's disease), anaphylaxis, allergic reaction, Sjögren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), male and female autoimmune infertility, ankylosing spondolytis, ulcerative colitis, mixed connective tissue disease, polyarteritis nedosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the autoimmune disease is a disorder of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjögren's syndrome, Hashimoto's thyroiditis, Graves' disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes. In some embodiments, the autoimmunie disorder is a T cell-mediated immunological disorder.

In some embodiments, the amount of the Conjugate administered ranges from about 0.01 to about 10 mg/kg per dose. In some embodiments, the amount of the Conjugate administered ranges from about 0.01 to about 5 mg/kg per dose. In some embodiments, the amount of the Conjugate administered ranges from about 0.05 to about 5 mg/kg per dose. In some embodiments, the amount of the Conjugate administered ranges from about 0.1 to about 5 mg/kg per dose. In some embodiments, the amount of the Conjugate administered ranges from about 0.1 to about 4 mg/kg per dose. In some embodiments, the amount of the Conjugate administered ranges from about 0.05 to about 3 mg/kg per dose. In some embodiments, the amount of the Conjugate administered ranges from about 0.1 to about 3 mg/kg per dose. In some embodiments, the amount of the Conjugate administered ranges from about 0.1 to about 2 mg/kg per dose.

Drug Loading

The drug loading (p) is the average number of PBD drugs per cell binding agent, e.g. antibody. Where the compounds of the invention are bound to cysteines, drug loading may range from 1 to 8 drugs (D) per cell binding agent, i.e. where 1, 2, 3, 4, 5, 6, 7, and 8 drug moieties are covalently attached to the cell binding agent. Compositions of conjgates include collections of cell binding agents, e.g. antibodies, conjugated with a range of drugs, from 1 to 8. Where the compounds of the invention are bound to lysines, drug loading may range from 1 to 80 drugs (D) per cell binding agent, although an upper limit of 40, 20, 10 or 8 may be preferred. Compositions of conjgates include collections of cell binding agents, e.g. antibodies, conjugated with a range of drugs, from 1 to 80, 1 to 40, 1 to 20, 1 to 10 or 1 to 8.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the Drug Linker. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of Drug Linker relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with linker reagents or the drug-linker reagents of the present invention which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the invention include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per cell binding agent is in the range 1 to 20. In some embodiments the range is selected from 1 to 8, 2 to 8, 2 to 6, 2 to 4, and 4 to 8.

In some embodiments, there is one dimer pyrrolobenzodiazepine group per cell binding agent.

### BRIEF DESCRIPTION OF FIGURE

FIG. 1 shows the effect of a conjugate of the invention on the growth of a tumour in vivo.

### GENERAL SYNTHETIC ROUTES

The synthesis of PBD compounds is extensively discussed in the following references, which discussions are incorporated herein by reference:

a) WO 00/12508 (pages 14 to 30);
b) WO 2005/023814 (pages 3 to 10);
c) WO 2004/043963 (pages 28 to 29); and
d) WO 2005/085251 (pages 30 to 39).

### Synthesis Route

Compounds of the present invention of formula I where R²¹ is not H or ═O can be synthesised from a compound of Formula 2:

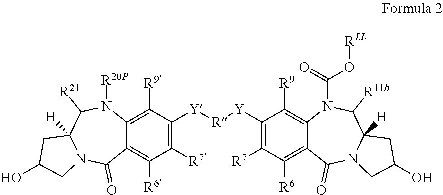

where R⁶, R⁷, R⁹, R^{6′}, R^{7′}, R^{9′}, R^{11b}, Y, Y′ and R″ are as defined for compounds of formula I, and R^{LL} is a protected version of R^{L}—this method is particularly applicable to compounds of formula I where R^{L} is of formula IIIa. R^{20P} is either R²⁰ or a precursor thereof. For these compounds, R^{LL} will typically be a portion of R^{L}, such as a group of formula IIIa′:

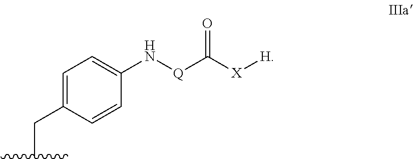

In such as case, the reaction involves adding the ground G_{L}. The precursor of R²⁰ may be of a similar structure, when R²⁰ is:

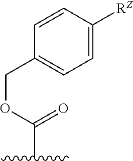

and R^{Z} is NH—C(═O)—X₁—NHC(═O)X₂—NH—R^{ZC}.

The compounds of Formula 2 may be made by deprotecting the R^{LL} group of compounds of Formula 3:

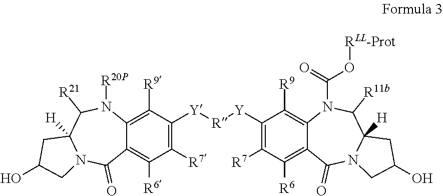

where R⁶, R⁷, R⁹, R^{6′}, R^{7′}, R^{9′}, R^{11b}, Y, Y′ and R″ are as defined for compounds of formula I, R^{LL-Prot} is a protected version of R^{LL}, and the Port^{N} represents a simple nitrogen protecting group (e.g. Fmoc, Boc) that is orthogonal to the R^{LL} protecting group. R^{20P} may be the same as the R^{20P} in Formula 2, or a protected version thereof, as appropriate.

Compounds of formula 3 may be made by ring-closure of compounds of Formula 4:

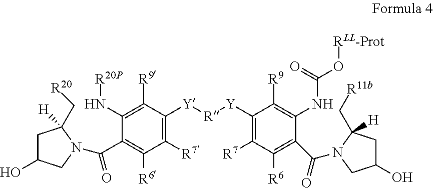

where the ring closure is carried out by oxidation, e.g. Swern.

Compounds of formula 4 can be synthesised from compounds of formula 5:

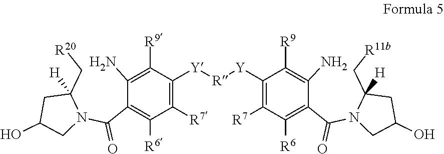

by a step-wise addition of two protecting groups. This can be achieved by simple protection of the amino group which will result in the imino bond in the final compound (e.g. by Fmoc, Boc), followed by installation of a desired protecting group at the other amino group.

Compounds of formula I where R^{L} is of formula IIIb, may be synthesised in a similar manner, although the complete R^{L} group may be installed starting from a compound of Formula 5, rather than with the use of a protected precursor.

Compounds of Formula 5 can be synthesised by known methods, such as those disclosed in WO 2011/130598.

Alternatively, compounds of Formula 4 can be synthesised by a monomeric route.

Compounds of the invention where R²¹ is H or ═O can be synthesised by a monomeric route, where the monomer containing these groups is full constructed before linking to the remainder of the compound. Reference is made to the routes shown in WO2014/096368.

Synthesis of Drug Conjugates

Conjugates can be prepared as previously described. Antibodies can be conjugated to the Drug Linker compound as described in Doronina et al., Nature Biotechnology, 2003, 21, 778-784). Briefly, antibodies (4-5 mg/mL) in PBS containing 50 mM sodium borate at pH 7.4 are reduced with tris(carboxyethyl)phosphine hydrochloride (TCEP) at 37° C. The progress of the reaction, which reduces interchain disulfides, is monitored by reaction with 5,5′-dithiobis(2-nitrobenzoic acid) and allowed to proceed until the desired level of thiols/mAb is achieved. The reduced antibody is then cooled to 0° C. and alkylated with 1.5 equivalents of maleimide drug-linker per antibody thiol. After 1 hour, the reaction is quenched by the addition of 5 equivalents of N-acetyl cysteine. Quenched drug-linker is removed by gel filtration over a PD-10 column. The ADC is then sterile-filtered through a 0.22 μm syringe filter. Protein concentration can be determined by spectral analysis at 280 nm and 329 nm, respectively, with correction for the contribution of drug absorbance at 280 nm. Size exclusion chromatography can be used to determine the extent of antibody aggregation, and RP-HPLC can be used to determine the levels of remaining NAC-quenched drug-linker.

Further Preferences

The following preferences may apply to all aspects of the invention as described above, or may relate to a single aspect. The preferences may be combined together in any combination.

In some embodiments, R^{6′}, R^{7′}, R^{9′}, and Y′ are selected from the same groups as R⁶, R⁷, R⁹, and Y respectively. In some of these embodiments, R^{6′}, R^{7′}, R^{9′}, and Y′ are the same as R⁶, R⁷, R⁹, and Y respectively.

N10′-C11′

In some embodiments, R²⁹ is H and R²¹ is H.

In some embodiments, R²⁹ is H and R²¹ is ═O.

In some embodiments, R²¹ is OH or OR^{A}, where R^{A} is C₁₋₄ alkyl and R²⁰ is selected from:

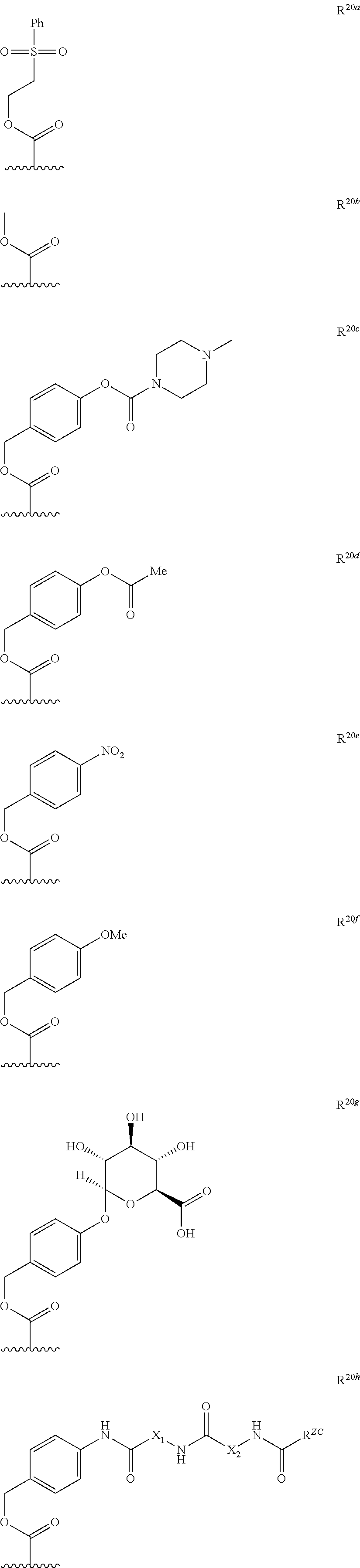

—C(═O)—X₁—NHC(═O)X₂—NH— represent a dipeptide. The amino acids in the dipeptide may be any combination of natural amino acids. The dipeptide may be the site of action for cathepsin-mediated cleavage.

In one embodiment, the dipeptide, —C(═O)—X₁—NHC(═O)X₂—NH—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-,
-Trp-Cit-
   where Cit is citrulline.

Preferably, the dipeptide, —C(═O)—X₁—NHC(═O)X₂—NH—, is selected from:
-Phe-Lys-,
-Val-Ala-,
-Val-Lys-,
-Ala-Lys-,
-Val-Cit-.

Most preferably, the dipeptide, —C(═O)—X₁—NHC(═O)X₂—NH—, is -Phe-Lys- or -Val-Ala-.

Other dipeptide combinations may be used, including those described by Dubowchik et al., Bioconjugate Chemistry, 2002, 13,855-869, which is incorporated herein by reference.

In one embodiment, the amino acid side chain is derivatised, where appropriate. For example, an amino group or carboxy group of an amino acid side chain may be derivatised.

In one embodiment, an amino group NH₂ of a side chain amino acid, such as lysine, is a derivatised form selected from the group consisting of NHR and NRR′.

In one embodiment, a carboxy group COOH of a side chain amino acid, such as aspartic acid, is a derivatised form selected from the group consisting of COOR, CONH₂, CONHR and CONRR′.

In one embodiment, the amino acid side chain is chemically protected, where appropriate. The side chain protecting group may be a group as discussed above. The present inventors have established that protected amino acid sequences are cleavable by enzymes. For example, it has been established that a dipeptide sequence comprising a Boc side chain-protected Lys residue is cleavable by cathepsin.

Protecting groups for the side chains of amino acids are well known in the art and are described in the Novabiochem Catalog. Additional protecting group strategies are set out in Protective Groups in Organic Synthesis, Greene and Wuts.

Possible side chain protecting groups are shown below for those amino acids having reactive side chain functionality:
Arg: Z, Mtr, Tos;
Asn: Trt, Xan;
Asp: Bzl, t-Bu;
Cys: Acm, Bzl, Bzl-OMe, Bzl-Me, Trt;
Glu: Bzl, t-Bu;
Gln: Trt, Xan;
His: Boc, Dnp, Tos, Trt;
Lys: Boc, Z—Cl, Fmoc, Z, Alloc;
Ser: Bzl, TBDMS, TBDPS;
Thr: Bz;
Trp: Boc;
Tyr: Bzl, Z, Z—Br.

In one embodiment, the side chain protection is selected to be orthogonal to a group provided as, or as part of, a capping group, where present. Thus, the removal of the side chain protecting group does not remove the capping group, or any protecting group functionality that is part of the capping group.

In other embodiments of the invention, the amino acids selected are those having no reactive side chain functionality. For example, the amino acids may be selected from: Ala, Gly, Ile, Leu, Met, Phe, Pro, and Val.

It is particularly preferred in the present invention, that if L¹ comprises a dipeptide, then —C(═O)—X₁—NHC(═O)X₂—NH— is the same dipeptide. An example of a preferred group is:

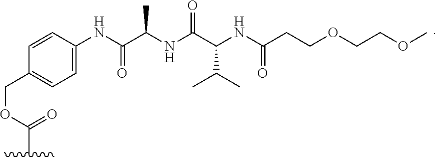 [SMILES: *C(=O)OCc1ccc(NC(=O)[C@@H](C)NC(=O)[C@H](NC(=O)CCOCCOC)C(C)C)cc1]

Other preferred R²⁰ groups include:

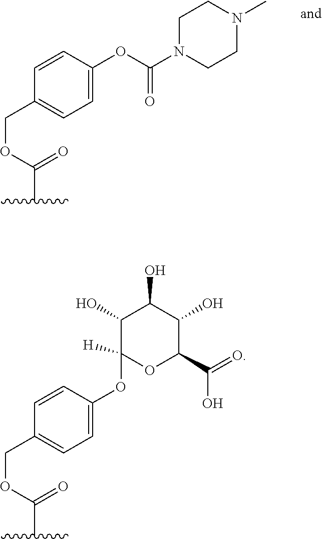 [SMILES: *C(=O)OCc1ccc(OC(=O)N2CCN(C)CC2)cc1 ; *C(=O)OCc1ccc(O[C@@H]2O[C@H](C(=O)O)[C@@H](O)[C@H](O)[C@H]2O)cc1]

Dimer Link

In some embodiments, Y and Y′ are both O.

In some embodiments, R″ is a C₃₋₇ alkylene group with no substituents. In some of these embodiments, R″ is a C₃, C₅ or C₇ alkylene. In particular, R″ may be a C₃ or C₅ alkylene.

In other embodiments, R″ is a group of formula:

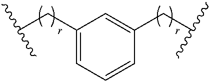

where r is 1 or 2.

The phenylene group may be replaced by a pyridylene group.

R⁶ to R⁹

In some embodiments, R⁹ is H.

In some embodiments, R⁶ is selected from H, OH, OR, SH, NH₂, nitro and halo, and may be selected from H or halo. In some of these embodiments R⁶ is H.

In some embodiments, R⁷ is selected from H, OH, OR, SH, SR, NH₂, NHR, NRR′, and halo. In some of these embodiments R⁷ is selected from H, OH and OR, where R is selected from optionally substituted C₁₋₇ alkyl, C₃₋₁₀ heterocyclyl and C₅₋₁₀ aryl groups. R may be more preferably a C₁₋₄ alkyl group, which may or may not be substituted. A substituent of interest is a C₅₋₆ aryl group (e.g. phenyl). Particularly preferred substituents at the 7-positions are OMe and OCH₂Ph. Other substituents of particular interest are dimethylamino (i.e. —NMe₂); —(OC₂H₄)_{q}OMe, where q is from 0 to 2; nitrogen-containing 06 heterocyclyls, including morpholino, piperidinyl and N-methyl-piperazinyl.

These embodiments and preferences apply to R^{9′}, R^{6′} and R^{7′} respectively.

R^{11b}

In some embodiments, R^{11b} is OH.

In some embodiments, R^{11b} is OR^{A}, where R^{A} is C₁₋₄ alkyl. In some of these embodiments, R^{A} is methyl.

In some embodiments of the first aspect of the present invention are of formula Ia, Ib or Ic:

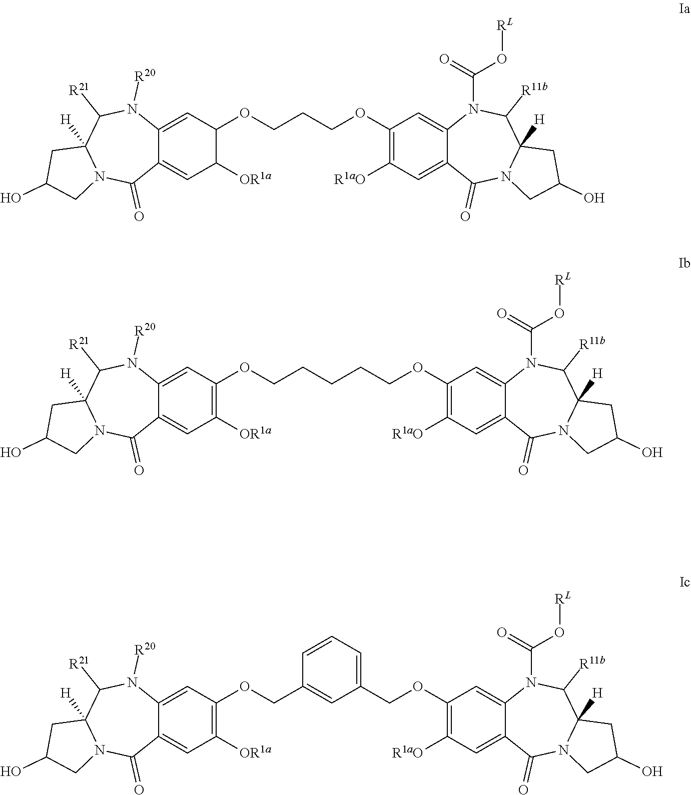

where R^{1a} is selected from methyl and benzyl;

R²⁰, R²¹, R^{L} and R^{11b} are as defined above.

These embodiments and preferences also apply to the second and fifth aspects of the invention.

Linker (R^{L})

In some embodiments, R^{L} is of formula IIIa.

In some embodiments, R^{LL} is of formula IIIa′.

G^{L}

G^{L} may be selected from

| | |
|---|---|
| 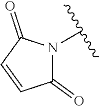 [SMILES: *N1C(=O)C=CC1=O] | (G^{L1-1}) |
| 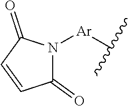 | (G^{L1-2}) |
| 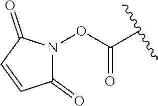 [SMILES: *C(=O)ON1C(=O)C=CC1=O] | (G^{L2}) |
| 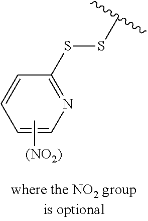 | (G^{L3-1}) |
| 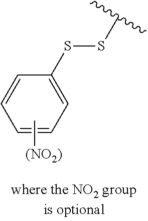 | (G^{L3-2}) |
| 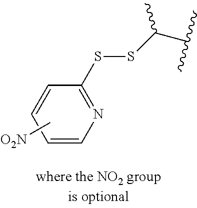 | (G^{L3-3}) |
| 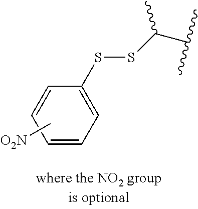 | (G^{L3-4}) |
| 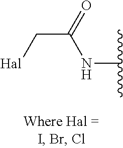 | (G^{L4}) |
| 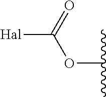 | (G^{L5}) |
| 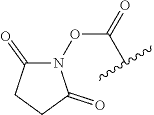 [SMILES: *C(=O)ON1C(=O)CCC1=O] | (G^{L6}) |
| 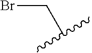 [SMILES: *CBr] | (G^{L7}) |
| 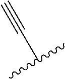 [SMILES: *C#C] | (G^{L8}) |
| 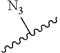 [SMILES: *N=[N+]=[N-]] | (G^{L9}) |

where Ar represents a C₅₋₆ arylene group, e.g. phenylene.

In some embodiments, G^{L} is selected from G^{L1-1} and G^{L1-2}. In some of these embodiments, G^{L} is G^{L1-1}.

G^{LL}

G^{LL} may be selected from:

| | |
|---|---|
| 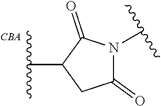 [SMILES: *C1CC(=O)N(*)C1=O] | (G^{LL1-1}) |
| 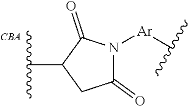 | (G^{LL1-2}) |
| 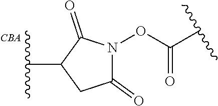 [SMILES: *C(=O)ON1C(=O)CC(*)C1=O] | (G^{LL2}) |
| 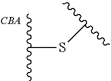 [SMILES: *S*] | (G^{LL3-1}) |
| 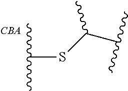 [SMILES: *SC(*)C] | (G^{LL3-2}) |
| 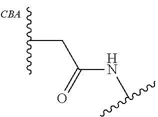 [SMILES: *CC(=O)N*] | (G^{LL4}) |
| 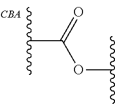 [SMILES: *OC(*)=O] | (G^{LL5}) |
| 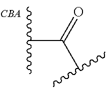 [SMILES: *C(*)=O] | (G^{LL6}) |
| 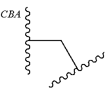 [SMILES: *C*] | (G^{LL7}) |
| 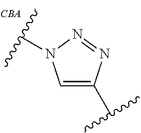 [SMILES: *c1cn(*)nn1] | (G^{LL8-1}) |
| 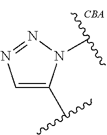 [SMILES: *c1cnnn1*] | (G^{LL8-2}) |
| 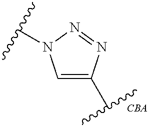 [SMILES: *c1cn(*)nn1] | (G^{LL9-1}) |
| 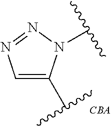 [SMILES: *c1cnnn1*] | (G^{LL9-2}) |

where Ar represents a C₅₋₆ arylene group, e.g. phenylene.

In some embodiments, G^{LL} is selected from G^{LL1-1} and G^{LL1-2}. In some of these embodiments, G^{LL} is G^{LL1-1}.

X

X is:

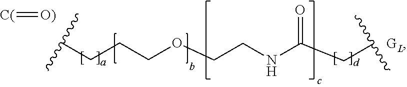

where a=0 to 5, b=0 to 16, c=0 or 1, d=0 to 5.
a may be 0, 1, 2, 3, 4 or 5. In some embodiments, a is 0 to 3. In some of these embodiments, a is 0 or 1. In further embodiments, a is 0.
b may be 0, 1, 2, 3, 4, 5, 6, 7, 8, 9, 10, 11, 12, 13, 14, 15 or 16. In some embodiments, b is 0 to 12. In some of these embodiments, b is 0 to 8, and may be 0, 2, 4 or 8.
c may be 0 or 1.
d may be 0, 1, 2, 3, 4 or 5. In some embodiments, d is 0 to 3. In some of these embodiments, d is 1 or 2. In further embodiments, d is 2.

In some embodiments of X, a is 0, c is 1 and d is 2, and b may be from 0 to 8. In some of these embodiments, b is 0, 4 or 8.

Q

In one embodiment, Q is an amino acid residue. The amino acid may a natural amino acids or a non-natural amino acid.

In one embodiment, Q is selected from: Phe, Lys, Val, Ala, Cit, Leu, Ile, Arg, and Trp, where Cit is citrulline.

In one embodiment, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q is selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{m},
^{CO}-Phe-Cit-^{NH},
^{CO}-Leu-Cit-^{NH},
^{CO}-Ile-Cit-^{NH},
^{CO}-Phe-Arg-^{NH}, and
^{CO}-Trp-Cit-^{NH};
   where Cit is citrulline.

Preferably, Q is selected from:
^{CO}-Phe-Lys-^{NH},
^{CO}-Val-Ala-^{NH},
^{CO}-Val-Lys-^{NH},
^{CO}-Ala-Lys-^{NH},
^{co}Val-Cit-^{NH}.

Most preferably, Q is selected from ^{co}-Phe-Lys-^{NH}, ^{co}-Val-Cit-^{NH} and ^{co}-Val-Ala-^{NH}.

Other dipeptide combinations of interest include:
^{CO}-Gly-Gly-^{NH},
^{CO}-Pro-Pro-^{NH}, and
^{CO}-Val-Glu-^{NH}.

Other dipeptide combinations may be used, including those described by Dubowchik et al., Bioconjugate Chemistry, 2002, 13,855-869, which is incorporated herein by reference.

In some embodiments, Q^{X} is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin.

In one embodiment, the amino acid side chain is chemically protected, where appropriate. The side chain protecting group may be a group as discussed below. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

In some embodiments, R^{L} is of formula IIIb.

In some embodiments, R^{LL} is of formula IIIb′.

R^{L1} and R^{L2} are independently selected from H and methyl, or together with the carbon atom to which they are bound form a cyclopropylene or cyclobutylene group.

In some embodiments, both R^{L1} and R^{L2} are H.

In some embodiments, R^{L1} is H and R^{L2} is methyl.

In some embodiments, both R^{L1} and R^{L2} are methyl.

In some embodiments, R^{L1} and R^{L2} together with the carbon atom to which they are bound form a cyclopropylene group.

In some embodiments, R^{L1} and R^{L2} together with the carbon atom to which they are bound form a cyclobutylene group.

In the group IIIb, in some embodiments, e is 0. In other embodiments, e is 1 and the nitro group may be in any available position of the ring. In some of these embodiments, it is in the ortho position. In others of these embodiments, it is in the para position.

In one particular embodiment, the first aspect of the invention comprises a compound of formula Id:

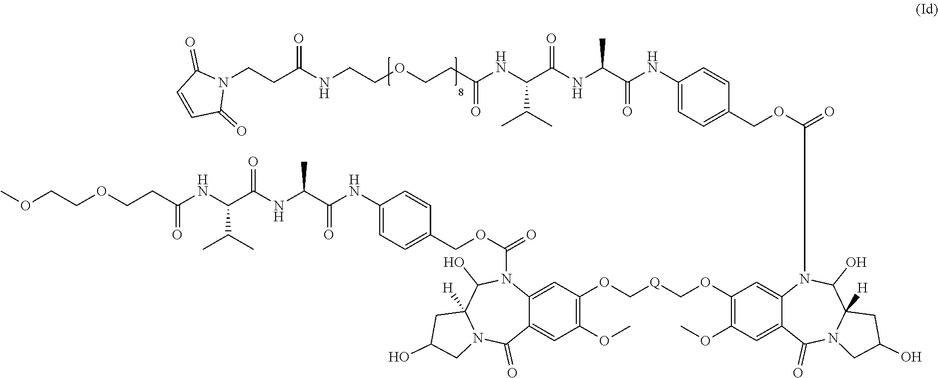

where Q is selected from:

### (a) —CH₂—;

(b) —C₃H₆—; and

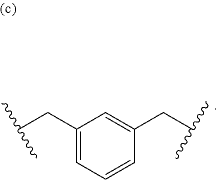 [SMILES: *Cc1cccc(C*)c1]

In one particular embodiment, the second aspect of the invention, the Drug linker (D^{L}) is of formula (Id′):

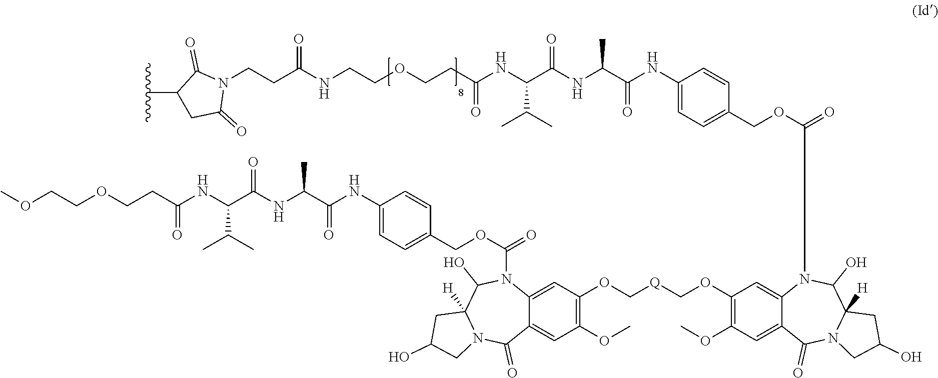

where Q is selected from:

### (a) —CH₂—;

(b) —C₃H₆—; and

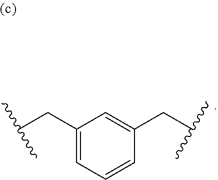 [SMILES: *Cc1cccc(C*)c1]

In some embodiments of the present invention, the C11 substituent may be in the following stereochemical arrangement relative to neighbouring groups:

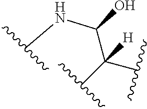

In other embodiments, the C11 substituent may be in the following stereochemical arrangement relative to neighbouring groups:

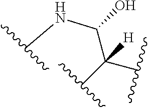

In some embodiments of the present invention, the C2 OH substituent may be in the following stereochemical arrangement relative to neighbouring groups:

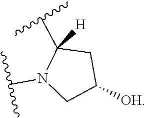 [SMILES: *[C@@H]1C[C@H](O)CN1*]

### EXAMPLES

### General Information

Flash chromatography was performed using a Biotage Isolera 1™ using gradient elution starting from either 88% hexane/EtOAc or 99.9% DCM/MeOH until all UV active components (detection at 214 and 254 nm) eluted from the column. The gradient was manually held whenever substantial elution of UV active material was observed. Fractions were checked for purity using thin-layer chromatography (TLC) using Merck Kieselgel 60 F254 silica gel, with fluorescent indicator on aluminium plates. Visualisation of TLC was achieved with UV light or iodine vapour unless otherwise stated. Extraction and chromatography solvents were bought and used without further purification from VWR U.K. All fine chemicals were purchased from Sigma-Aldrich or TCI Europe unless otherwise stated. Pegylated reagents were obtained from Quanta biodesign US via Stratech UK.

The analytical LC/MS conditions (for reaction monitoring and purity determination) were as follows: Positive mode electrospray mass spectrometry was performed using a Shimadzu Nexera®/Prominence® LCMS-2020. Mobile phases used were solvent A (H₂O with 0.1% formic acid) and solvent B (CH₃CN with 0.1% formic acid). Gradient for routine 3-minute run: Initial composition 5% B held over 25 seconds, then increased from 5% B to 100% B over a 1 minute 35 second period. The composition was held for 50 seconds at 100% B, then returned to 5% B in 5 seconds and held there for 5 seconds. The total duration of the gradient run was 3.0 minutes. Gradient for 15-minute run: Initial composition 5% B held over 1 minute, then increased from 5% B to 100% B over a 9 minute period. The composition was held for 2 minutes at 100% B, then returned to 5% B in 10 seconds and held there for 2 minutes 50 seconds. The total duration of the gradient run was 15.0 minutes. Flow rate was 0.8 mL/minute (for 3-minute run) and 0.6 mL/minute (for 15-minute run). Detection was at 254 nm. Columns: Waters Acquity UPLC® BEH Shield RP18 1.7 μm 2.1×50 mm at 50° C. fitted with Waters Acquity UPLC® BEH Shield RP18 VanGuard Pre-column, 130A, 1.7 μm, 2.1 mm×5 mm (routine 3-minute run); and ACE Excel 2 C18-AR, 2μ, 3.0×100 mm fitted with Waters Acquity UPLC® BEH Shield RP18 VanGuard Pre-column, 130A, 1.7 μm, 2.1 mm×5 mm (15-minute run).

The preparative HPLC conditions were as follows: Reverse-phase ultra-fast high-performance liquid chromatography (UFLC) was carried out on a Shimazdzu Prominence® machine using a Phenomenex® Gemini NX 5μ C18 column (at 50° C.) 150×21.2 mm. Eluents used were solvent A (H₂O with 0.1% formic acid) and solvent B (CH₃CN with 0.1% formic acid). All UFLC experiments were performed with gradient conditions: Initial composition 13% B increased to 60% B over a 15 minute period then increased to 100% B over 2 minutes. The composition was held for 1 minute at 100% B, then returned to 13% B in 0.1 minute and held there for 1.9 minutes. The total duration of the gradient run was 20.0 minutes. Flow was 20.0 mL/minute and detection was at 254 and 280 nm.

### Example 1

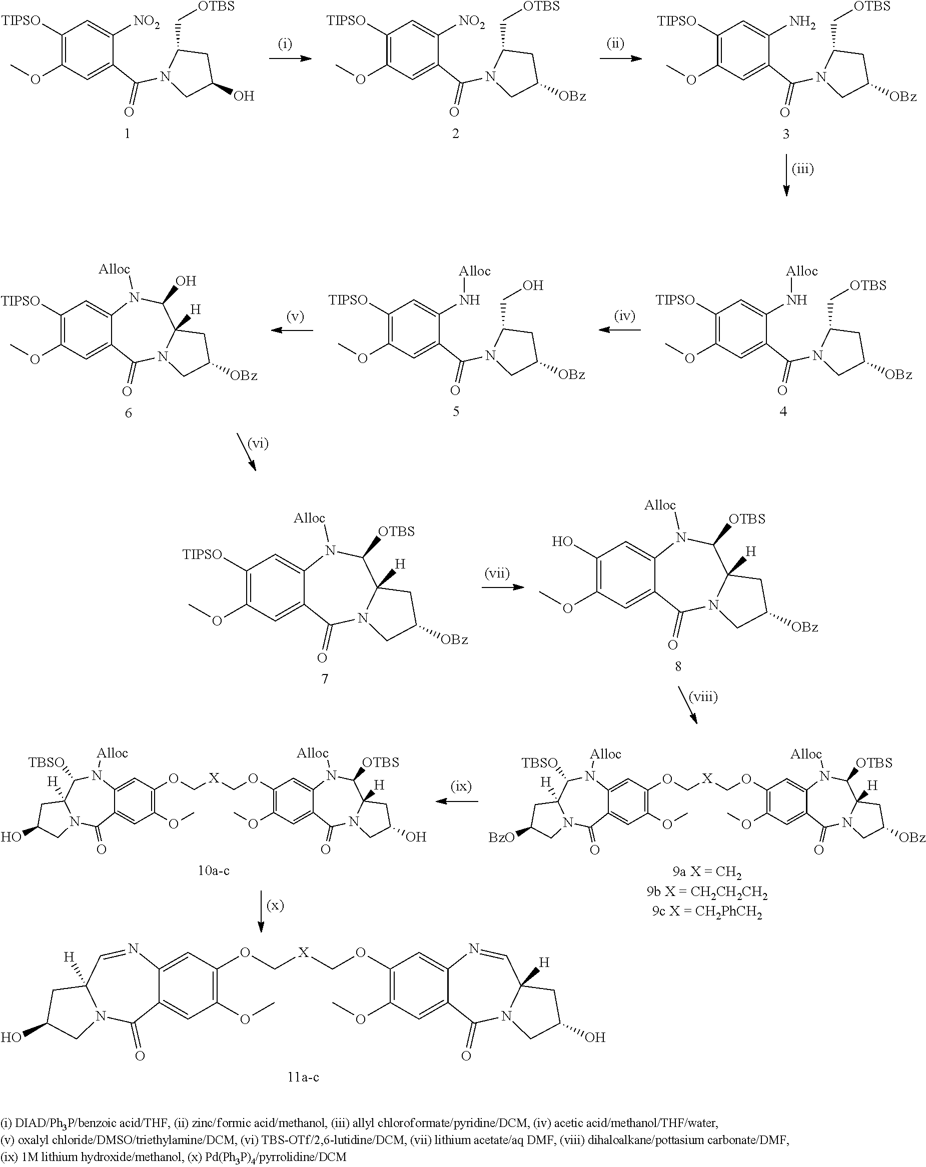

### a) (3S,5S)-5-(((tert-butyldimethylsilyl)oxy)methyl)-1-(5-methoxy-2-nitro-4-((triisopropylsilyl)oxy) benzoyl)pyrrolidin-3-yl benzoate 2

Diethylazodicarboxylate (17.34 g, 0.085 mol, 5.0 eq) was added to a solution of triphenylphosphine (22.49 g, 0.085 mol, 5.0 eq) in THF (300 mL) and stirred at room temperature for 30 min. 1 (10 g, 0.017 mol, 1.0 eq) was added and stirring continued for a further 30 min, until a white ppt had formed. Benzoic acid (2.1 g, 0.017 mol, 1.0 eq) was added, the ppt turned from white to orange and then back to white. After 30 min, the ppt was removed by filtration. The filtrate was evaporated to dryness and purified by flash chromatography (10% ethyl acetate/heptane to remove the excess mitsunobu reagents followed by 20% ethyl acetate/heptane to elute the product as a white solid). Yield=9.5 g (81%). LC/MS rt 2.28 min m/z (687.4) M+H.

### b) (3S,5S)-1-(2-amino-5-methoxy-4-((triisopropylsilyl)oxy)benzoyl)-5-(((tert-butyldimethylsilyl)oxy)methyl)pyrrolidin-3-yl benzoate 3

Zinc dust (18.0 g, 0.27 mol, 20 eq) was added to a solution of 2 in methanol (75 mL) and stirred at room temperature. Formic acid (15 mL) was added which resulted in an exotherm of 35° C. After 10 mins, the zinc was removed by filtering through a short bed of celite, which was then washed with ethyl acetate (250 mL). The combined organic fractions were washed with saturated NaHCO₃ (100 mL) then brine (50 mL). The resulting organic phase was dried (MgSO₄) and evaporated under reduced pressure to leave a yellow residue which was purified by flash chromatography (gradient ethyl acetate/heptane, 15/85 to 20/80 v/v) to yield 3 as a colourless oil, 8.3 g (91%). LC/MS rt 2.24 min m/z (657.3) M+H.

### c) (3S,5S)-1-(2-(((allyloxy)carbonyl)amino)-5-methoxy-4-((triisopropylsilyl)oxy)benzoyl)-5-(((tert-butyldimethylsilyl)oxy)methyl)pyrrolidin-3-yl benzoate 4

Allyl chloroformate (1.65 g, 13.7 mmol, 1.1 eq) was added dropwise to a solution of 3 (8.2 g, 12.4 mmol, 1.0 eq) and pyridine (1.48 g, 18.7 mmol, 5.0 eq) in dichloromethane (75 mL) at 5° C. The reaction mixture was allowed to warm to room temperature and stirred for a further 60 mins. The organic phase was washed successively with 0.1M HCl (20 mL), saturated sodium hydrogen carbonate (20 mL) and brine (10 mL). After drying (MgSO₄), the solvent was removed under reduced pressure to leave a white solid which was used in the next step without further purification, 8.5 g (92%).

### d) (3S,5S)-1-(2-(((allyloxy)carbonyl)amino)-5-methoxy-4-((triisopropylsilyl)oxy)benzoyl)-5-(hydroxymethyl)pyrrolidin-3-yl benzoate 5

4 (8.5 g, 11.5 mmol) was dissolved in a mixture of acetic acid (35 mL), methanol (5 mL), THF (5 mL) and water (10 mL). The resulting solution was stirred at room temperature overnight. The reaction mixture was then poured into ethyl acetate (100 mL) and washed successively with water (2×100 mL), saturated sodium hydrogen carbonate (50 mL) and brine (50 mL). After drying (MgSO₄), the solvent was removed under reduced pressure and the residue purified by flash chromatography (gradient ethyl acetate/heptane, 40/60 to 50/50 v/v) to yield 5 as a white solid, 5.24 g (73%). LC/MS rt 1.98 min m/z (627.5) M+H.

### e) Allyl (2S,11S,11aS)-2-(benzoyloxy)-11-hydroxy-7-methoxy-5-oxo-8-((triisopropylsilyl)oxy)-2,3,11,11a-tetrahydro-1H-benzo[e]pyrrolo[1,2-a][1,4]diazepine-10(5H)-carboxylate 6

Oxalyl chloride (2M in DCM, 4.6 mL, 9.20 mmol, 1.1 eq) was added dropwise to a solution of DMSO (1.63 g, 20.9 mmol, 2.5 eq) in dry dichloromethane (75 mL) at −78° C. under an Argon atmosphere. After 15 mins, a solution of 5 (5.24 g, 8.36 mmol, 1.0 eq) in dichloromethane (20 mL) was added dropwise and the reaction mixture stirred for a further 30 mins. Triethyl amine (4.2 g, 41.8 mmol, 5.0 eq) was added and the resulting solution allowed to warm to room temperature and then stirred for a further 60 mins. The organic phase was then washed successively with 0.1M HCl (25 mL), saturated sodium hydrogen carbonate (25 mL) and brine (10 mL). After drying (MgSO₄), the solvent was removed under reduced pressure to leave a white solid which was used in the next step without further purification, 4.52 g (87%). LC/MS rt 1.90 min m/z (625.3) M+H.

### f) Allyl (2S,11S,11aS)-2-(benzoyloxy)-11-((tert-butyldimethylsilyl)oxy)-7-methoxy-5-oxo-8-((triisopropylsilyl)oxy)-2,3,11,11a-tetrahydro-1H-benzo[e]pyrrolo[1,2-a][1,4]diazepine-10(5H)-carboxylate 7

tert-Butyldimethylsilyl trifluoromethanesulfonate (5.7 g, 21.6 mmol, 3.0 eq) was added dropwise to a solution of 6 (4.5 g, 7.2 mmol, 1.0 eq) and 2,6-lutidine (3.1 g, 28.8 mmol, 4.0 eq) in dichloromethane (100 mL) at 5° C. under an atmosphere of Argon. The reaction mixture was allowed to warm to room temperature and stirred for a further 3 hrs. The organic layer was then washed successively with water (25 mL), saturated sodium hydrogen carbonate (25 mL) and brine (15 mL). After drying (MgSO₄), the solvent was removed under reduced pressure and the residue purified by flash chromatography (ethyl acetate/heptane, 50/50 v/v) to yield 7 as a white solid, 4.0 g (76%). LC/MS rt 2.29 min m/z (739.3) M+H.

### g) Allyl (2S,11S,11aS)-2-(benzoyloxy)-11-((tert-butyldimethylsilyl)oxy)-8-hydroxy-7-methoxy-5-oxo-2,3,11,11a-tetrahydro-1H-benzo[e]pyrrolo[1,2-a][1,4]diazepine-10(5H)-carboxylate 8

Lithium acetate dihydrate (0.55 g, 5.4 mmol, 1.0 eq) was added to a solution of 7 (4.0 g, 5.4 mmol, 1.0 eq) in DMF/water (98/2, 5 mL) and stirred at room temperature for 5 hrs. The reaction mixture was diluted with ethyl acetate (100 mL) and the organic phase washed successively with 1M citric acid (50 mL) and brine (50 mL). After drying (MgSO₄), the solvent was removed under reduced pressure and the residue purified by flash chromatography (gradient ethyl acetate/heptane, 75/25 to 100/0 v/v) to leave 8 as a white solid, 3.0 g (95%). LC/MS rt 1.80 min m/z (583.4) M+H.

### h) General Method for the Dimerisation of 8 to 9

Potassium carbonate (2.5 eq) was added to a solution of 8 (1.0 g, 1.72 mmol, 2.1 eq) and either 1,3-dibromopropane; 1,5-diiodopentane; or 1,3-bis(bromomethyl)benzene (1.0 eq) in DMF (5 mL). The resulting mixture was stirred at 75° C. for 3 days. After diluting with dichloromethane (25 mL), the inorganics were removed by filtration and the filtrate evaporated to dryness under reduced pressure. The residue was purified by flash chromatography to leave the products as white solids.

### i) diallyl 8,8′-(propane-1,3-diylbis(oxy))(2S,2′S,11S,11aS,11′S,11a′S)-bis(2-(benzoyloxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate) 9a

(gradient: ethyl acetate/heptane, 50/50 to 100/0 v/v). Yield 0.88 g (90%). LC/MS rt 2.17 min m/z (1227.4) M+Na.

### ii) diallyl 8,8′-(pentane-1,5-diylbis(oxy))(2S,2′S,11S,11aS,11′S,11a′S)-bis(2-(benzoyloxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate) 9b

(ethyl acetate). Yield 0.82 g (82%). LC/MS rt 2.20 min m/z (1255.3) M+Na. diallyl 8,8′-((1,3-phenylenebis(methylene))bis(oxy))(2S,2′S,11S,11aS,11′S,11a′S)-bis(2-(benzoyloxy)-11-((tert-butyldimethylsilyl)oxy)-7-methoxy-5-oxo-2,3,11,11a-tetrahydro-1H—

### iii) benzo[e]pyrrolo[1,2-a][1,4]diazepine-10(5H)-carboxylate) 9c

(gradient: ethyl acetate/heptane, 75/25 to 100/0 v/v). Yield 0.9 g (85%). LC/MS rt 2.20 min m/z (1267.8) M+H.

### i) General Method for Ester Hydrolysis of 9 to 10

1M lithium hydroxide (1 mL) was added to a solution of 9 in methanol (10 mL) and stirred at room temperature for 2 hrs. The methanol was then removed under reduced pressure and the remaining aqueous layer acidified (pH 6) with 1M citric acid. The product was extracted into ethyl acetate (30 mL), dried (MgSO₄) and evaporated under reduced pressure to leave a white solid which was purified by flash chromatography.

### i) diallyl 8,8′-(propane-1,3-diylbis(oxy))(2S,2′S,11S,11aS,11′S,11a′S)-bis(11-((tert-butyldimethylsilyl)oxy)-2-hydroxy-7-methoxy-5-oxo-2,3,11,11a-tetrahydro-1H-benzo[e]pyrrolo[1,2-a][1,4]diazepine-10(5H)-carboxylate) 10a

(gradient: methanol/dichloromethane, 2/98 to 4/96 v/v). Yield 0.64 g (89%). LC/MS rt 1.86 min m/z (997.4) M+H.

### ii) diallyl 8,8′-(pentane-1,5-diylbis(oxy))(2S,2′S,11S,11aS,11′S,11a′S)-bis(11-((tert-butyldimethylsilyl)oxy)-2-hydroxy-7-methoxy-5-oxo-2,3,11,11a-tetrahydro-1H-benzo[e]pyrrolo[1,2-a][1,4]diazepine-10(5H)-carboxylate) 10b

(methanol/dichloromethane, 5/95 v/v). Yield 0.68 g (93%). LC/MS rt 1.91 min m/z (1025.7) M+H.

### iii) diallyl 8,8′-((1,3-phenylenebis(methylene))bis(oxy))(2S,2′S,11S,11aS,11′S,11a′S)-bis(11-((tert-butyldimethylsilyl)oxy)-2-hydroxy-7-methoxy-5-oxo-2,3,11,11a-tetrahydro-1H-benzo[e]pyrrolo[1,2-a][1,4]diazepine-10(5H)-carboxylate) 10c

(gradient: methanol/dichloromethane, 2/98 to 4/96 v/v). Yield 0.54 g (74%). LC/MS rt 1.92 min m/z (1060.5) M+H.

### j) General Method Alloc/TBS Deprotection of 10 to 11

Tetrakis triphenylphosphine palladium(0) (2 mol %) was added to a solution of 10 (1.0 eq) and pyrrolidine (2.5 eq) in dichloromethane and stirred at room temperature for 30 min. The reaction mixture was diluted with dichloromethane and washed with saturated ammonium chloride. The organic phase was dried (MgSO₄) and the solvent removed under reduced pressure. The residue was purified by reverse phase HPLC to leave the product as a white solid.

### i) (2S,2′S,11aS,11a′S)-8,8′-(propane-1,3-diylbis(oxy))bis(2-hydroxy-7-methoxy-1,2,3,11a-tetrahydro-5H-benzo[e]pyrrolo[1,2-a][1,4]diazepin-5-one) 11a

LC/MS rt 3.84 min m/z (565.3) M+H.

### ii) (2S,2′S,11aS,11a′S)-8,8′-(pentane-1,5-diylbis(oxy))bis(2-hydroxy-7-methoxy-1,2,3,11a-tetrahydro-5H-benzo[e]pyrrolo[1,2-a][1,4]diazepin-5-one) 11b

LC/MS rt 1.12 min m/z (593.3) M+H.

### iii) (2S,2′S,11aS,11a′S)-8,8′-((1,3-phenylenebis(methylene))bis(oxy))bis(2-hydroxy-7-methoxy-1,2,3,11a-tetrahydro-5H-benzo[e]pyrrolo[1,2-a][1,4]diazepin-5-one) 11c

LC/MS rt 4.51 min m/z (626.7) M+H.

### Example 2

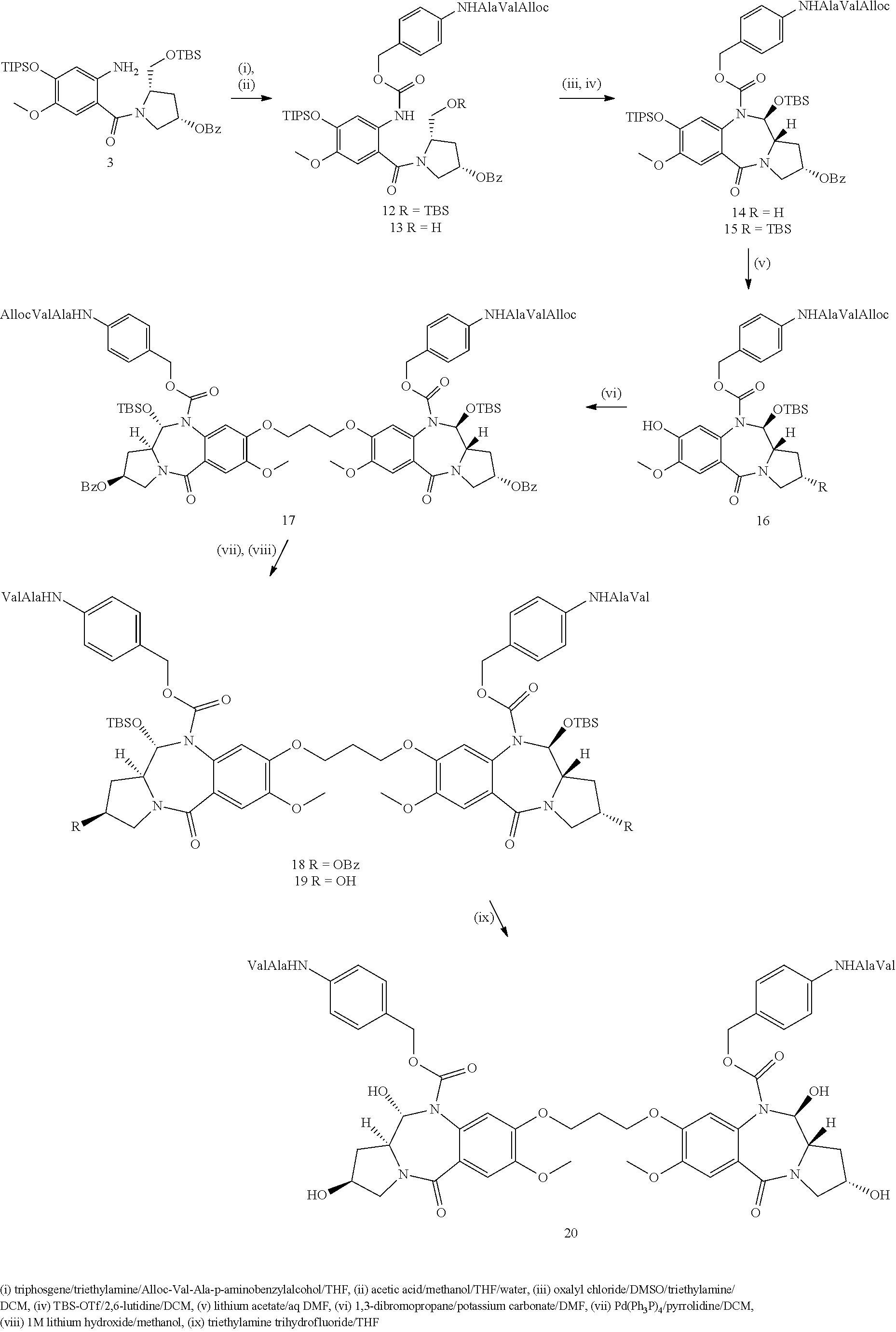

### a) (3S,5S)-1-(2-((((4-((R)-2-((R)-2-(((allyloxy)carbonyl)amino)-3-methylbutanamido) propanamido)benzyl)oxy)carbonyl)amino)-5-methoxy-4-((triisopropylsilyl)oxy)benzoyl)-5-(((tert-butyldimethylsilyl)oxy)methyl)pyrrolidin-3-yl benzoate 12

Triethylamine (1.35 g, 13.4 mmol, 2.2 eq) was added to a solution of 3 (4.0 g, 6.0 mmol, 1.0 eq) and triphosgene (0.65 g, 2.2 mmol, 0.36 eq) in THF (40 mL) and stirred a room temperature, under an atmosphere of N₂, for 5 min. A suspension of Alloc-Val-Ala-p-aminobenzyl alcohol (2.75 g, 7.3 mmol, 1.2 eq) and triethylamine (0.92 g, 9.1 mmol, 1.5 eq) in THF (25 mL) was added and the resulting mixture heated at 40° C. for 2 hr. The reaction mixture was filtered, the filtrate evaporated to dryness and purified by flash chromatography (methanol/dichloromethane, 2/98 v/v) to yield 12 as a pale yellow solid, 5.0 g (78%). LC/MS rt 2.24 min m/z (1082.4) M+Na.

### b) (3S,5S)-1-(2-((((4-((R)-2-((R)-2-(((allyloxy)carbonyl)amino)-3-methylbutanamido) propanamido)benzyl)oxy)carbonyl)amino)-5-methoxy-4-((triisopropylsilyl)oxy)benzoyl)-5-(hydroxymethyl)pyrrolidin-3-yl benzoate 13

12 (8.0 g, 7.5 mmol) was dissolved in a mixture of acetic acid (35 mL), methanol (5 mL), THF (5 mL) and water (10 mL). The resulting solution was stirred at room temperature overnight. The reaction mixture was then poured into ethyl acetate (100 mL) and washed successively with water (2×100 mL), saturated sodium hydrogen carbonate (50 mL) and brine (50 mL). After drying (MgSO₄), the solvent was removed under reduced pressure and the residue purified by flash chromatography (methanol/dichloromethane, 3/97 v/v) to yield 13 as a white solid, 5.6 g (79%). LC/MS rt 1.95 min m/z (946.3) M+H.

### c) 4-((R)-2-((R)-2-(((allyloxy)carbonyl)amino)-3-methylbutanamido)propanamido)benzyl (2S,11S)-2-(benzoyloxy)-11-hydroxy-7-methoxy-5-oxo-8-((triisopropylsilyl)oxy)-2,3,11,11a-tetrahydro-1H-benzo[e]pyrrolo[1,2-a][1,4]diazepine-10(5H)-carboxylate 14

Oxalyl chloride (2M in DCM, 0.83 mL, 1.6 mmol, 1.1 eq) was added dropwise to a solution of DMSO (0.27 mL, 3.7 mmol, 2.5 eq) in dry dichloromethane (20 mL) at −78° C. under an Argon atmosphere. After 15 mins, a solution of 13 (1.43 g, 1.5 mmol, 1.0 eq) in dichloromethane (5 mL) was added dropwise and the reaction mixture stirred for a further 30 mins. Triethyl amine (1.05 mL, 7.5 mmol, 5.0 eq) was added and the resulting solution allowed to warm to room temperature and then stirred for a further 60 mins. The organic phase was then washed successively with 0.1M HCl (15 mL), saturated sodium hydrogen carbonate (15 mL) and brine (10 mL). After drying (MgSO₄), the solvent was removed under reduced pressure and purified by flash chromatography (methanol/dichloromethane 2/98 v/v) to yield 14 as a white solid, 1.1 g (77%). LC/MS rt 1.86 min m/z (944.3) M+H.

### d) 4-((R)-2-((R)-2-(((allyloxy)carbonyl)amino)-3-methylbutanamido)propanamido)benzyl (2S,11S)-2-(benzoyloxy)-11-((tert-butyldimethylsilyl)oxy)-7-methoxy-5-oxo-8-((triisopropylsilyl)oxy)-2,3,11,11a-tetrahydro-1H-benzo[e]pyrrolo[1,2-a][1,4]diazepine-10(5H)-carboxylate 15

tert-Butyldimethylsilyl trifluoromethanesulfonate (0.92 g, 3.5 mmol, 3.0 eq) was added dropwise to a solution of 14 (1.1 g, 1.16 mmol, 1.0 eq) and 2,6-lutidine (0.5 g, 4.7 mmol, 4.0 eq) in dichloromethane (15 mL) at 5° C. under an atmosphere of Argon. The reaction mixture was allowed to warm to room temperature and stirred for a further 3 hrs. The organic layer was then washed successively with water (25 mL), saturated sodium hydrogen carbonate (25 mL) and brine (15 mL). After drying (MgSO₄), the solvent was removed under reduced pressure and the residue used in the next step without further purification, 1.2 g (97%). LC/MS rt 2.20 min m/z (1058.4) M+H.

### e) 4-((R)-2-((R)-2-(((allyloxy)carbonyl)amino)-3-methylbutanamido)propanamido)benzyl (2S,11S)-2-(benzoyloxy)-11-((tert-butyldimethylsilyl)oxy)-8-hydroxy-7-methoxy-5-oxo-2,3,11,11a-tetrahydro-1H-benzo[e]pyrrolo[1,2-a][1,4]diazepine-10(5H)-carboxylate 16

Lithium acetate dihydrate (0.11 g, 1.04 mmol, 1.0 eq) was added to a solution of 15 (1.1 g, 1.04 mmol, 1.0 eq) in DMF/water (98/2, 3 mL) and stirred at room temperature for 5 hrs. The reaction mixture was diluted with ethyl acetate (25 mL) and the organic phase washed successively with 1M citric acid (20 mL) and brine (20 mL). After drying (MgSO₄), the solvent was removed under reduced pressure and the residue purified by flash chromatography (gradient methanol/dichloromethane, 1/99 to 3/97 v/v) to leave 16 as a white solid, 0.82 g (85%). LC/MS rt 1.77 min m/z (902.3) M+H.

### f) 4-((R)-2-((R)-2-(((allyloxy)carbonyl)amino)-3-methylbutanamido)propanamido)benzyl (2S,11S,11aS)-8-(3-(((2S,11S,11aS)-10-(((4-((S)-2-((S)-2-(((allyloxy)carbonyl)amino)-3-methylbutanamido)propanamido)benzyl)oxy)carbonyl)-2-(benzoyloxy)-11-((tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)propoxy)-2-(benzoyloxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate 17

Potassium carbonate (0.18 g, 1.3 mmol, 2.5 eq) was added to a solution of 8 (1.0 g, 1.1 mmol, 2.1 eq) and 1,3-dibromopropane (0.1 g, 0.05 mmol, 1.0 eq) in DMF (5 mL). The resulting mixture was stirred at 75° C. for 3 days. After diluting with dichloromethane (25 mL), the inorganics were removed by filtration and the filtrate evaporated to dryness under reduced pressure. The residue was purified by flash chromatography (gradient methanol/dichloromethane, 2/98 to 4/96 v/v) to leave 17 as a white solid, 0.77 g (79%). LC/MS rt 2.04 min m/z (1844.3) M+H.

### g) 4-((R)-2-((R)-2-amino-3-methylbutanamido)propanamido)benzyl (2S,11S,11aS)-8-(3-(((2S,11S,11aS)-10-(((4-((S)-2-((S)-2-amino-3-methylbutanamido)propanamido) benzyl)oxy)carbonyl)-2-(benzoyloxy)-11-((tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)propoxy)-2-(benzoyloxy)-11-((tert-butyldimethylsilyl)oxy)-7-methoxy-5-oxo-2,3,11,11a-tetrahydro-1H-benzo[e]pyrrolo[1,2-a][1,4]diazepine-10(5H)-carboxylate 18

Pd(Ph₃P)₄ (21 mg, 5 mol %) was added to a solution of 17 (0.65 g, 0.35 mmol, 1.0 eq) and pyrrolidine (0.15 g, 2.1 mmol, 6.0 eq) in dichloromethane (10 mL) and stirred at room temperature for 60 mins. The reaction mixture was evaporated to dryness and purified by flash chromatography (gradient methanol/dichloromethane, 5/95 to 20/80 v/v) to leave 18 as a white solid, 0.55 g (93%). LC/MS rt 1.39 min m/z (1676.5) M+H.

### h) 4-((R)-2-((R)-2-amino-3-methylbutanamido)propanamido)benzyl (2S,11S,11aS)-8-(3-(((2S,11S,11aS)-10-(((4-((S)-2-((S)-2-amino-3-methylbutanamido)propanamido)benzyl)oxy) carbonyl)-11-((tert-butyldimethylsilyl)oxy)-2-hydroxy-7-methoxy-5-oxo-2,3,5,10,11,11a-hexahydro-1H-benzo[e]pyrrolo[1,2-a][1,4]diazepin-8-yl)oxy)propoxy)-11-((tert-butyldimethyl silyl)oxy)-2-hydroxy-7-methoxy-5-oxo-2,3,11,11a-tetrahydro-1H-benzo[e]pyrrolo[1,2-a] [1,4]diazepine-10(5H)-carboxylate 19

1M lithium hydroxide (0.5 mL) was added to a solution of 18 (250 mg, 0.15 mmol) in methanol (3 mL) and stirred at room temperature for 4 hrs. The methanol was removed under reduced pressure and the aqueous phase acidified (pH 4) with 1M citric acid. The resulting solution was purified by reverse phase isolera (acetonitrile/water, 35/65 v/v+0.1% formic acid) to leave 19 as a white solid, 156 mg (71%). LC/MS rt 1.24 min m/z (1468.2) M+H.

### i) 4-((R)-2-((R)-2-amino-3-methylbutanamido)propanamido)benzyl (2S,11S,11aS)-8-(3-(((2S,11S,11aS)-10-(((4-((S)-2-((S)-2-amino-3-methylbutanamido)propanamido)benzyl)oxy) carbonyl)-2,11-dihydroxy-7-methoxy-5-oxo-2,3,5,10,11,11a-hexahydro-1H-benzo[e]pyrrolo [1,2-a][1,4]diazepin-8-yl)oxy)propoxy)-2,11-dihydroxy-7-methoxy-5-oxo-2,3,11,11a-tetrahydro-1H-benzo[e]pyrrolo[1,2-a][1,4]diazepine-10(5H)-carboxylate 20

Triethylamine trihydrofluoride (96 mg, 0.59 mmol, 5.0 eq) was added to a solution of 19 (175 mg, 0.12 mmol, 1.0 eq) in THF (10 mL) and stirred at room temperature for 5 days. The solvent was removed under vacuum and the residue purified by prep HPLC to leave 20 as a white solid, 91 mg (62%). LC/MS rt 0.95 min m/z (1239.9) M+H.

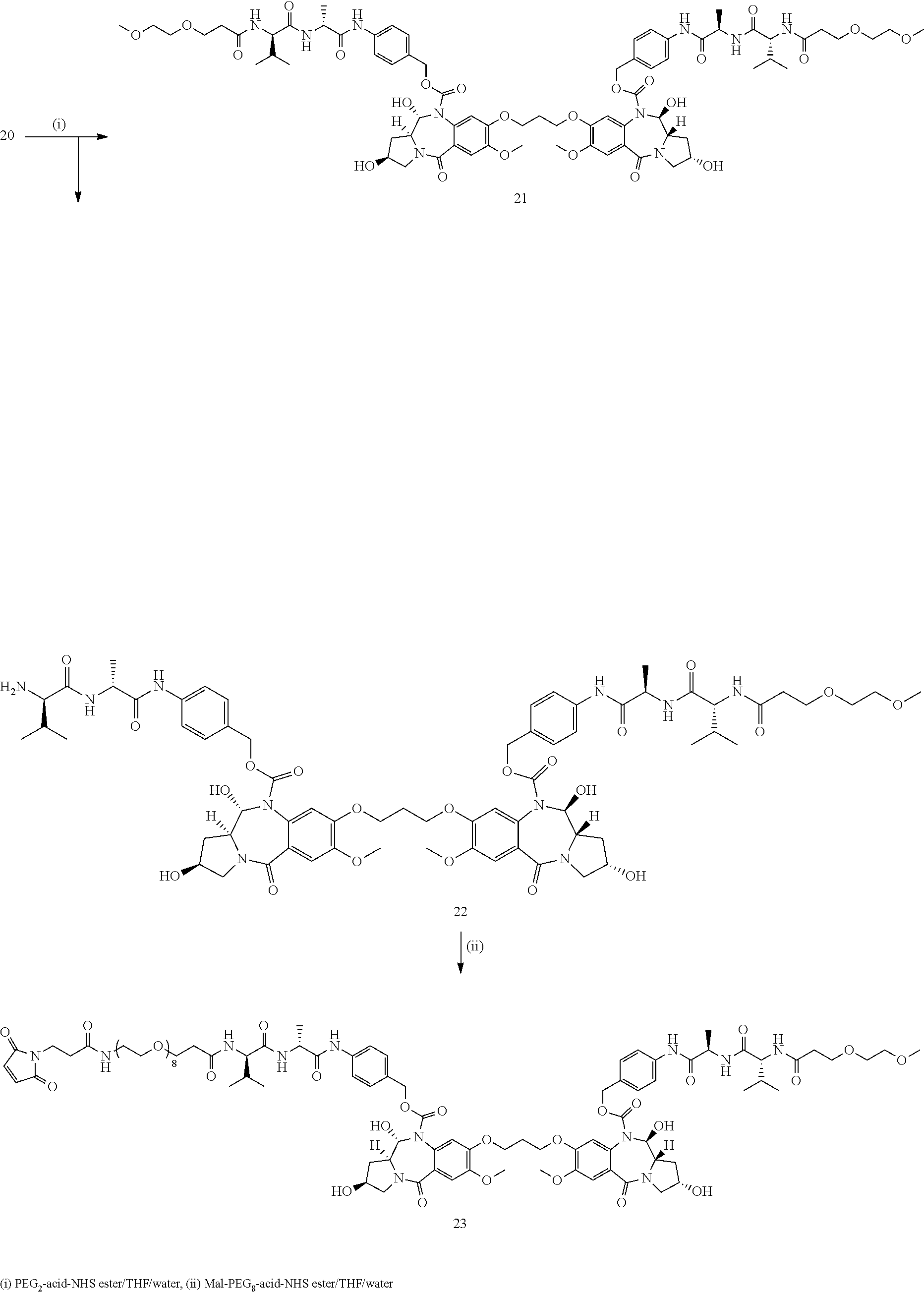

### j) 4-((R)-2-((R)-2-amino-3-methylbutanamido)propanamido)benzyl (2S,11S,11aS)-8-(3-(((2S,11S,11aS)-2,11-dihydroxy-10-(((4-((10R,13R)-10-isopropyl-13-methyl-8,11-dioxo-2,5-dioxa-9,12-diazatetradecan-14-amido)benzyl)oxy)carbonyl)-7-methoxy-5-oxo-2,3,5,10,11,11a-hexahydro-1H-benzo[e]pyrrolo[1, 2-a][1, 4]diazepin-8-yl)oxy)propoxy)-2,11-dihydroxy-7-methoxy-5-oxo-2,3,11,11a-tetrahydro-1H-benzo[e]pyrrolo[1,2-a][1,4]diazepine-10(5H)-carboxylate 22

Sodium hydrogen carbonate (6 mg, 0.07 mmol, 1.05 eq) was dissolved in water (0.5 mL) and added to a solution of 20 (91 mg, 0.07 mmol, 1.0 eq) in THF (0.5 mL). PEG₂-COOH NHS ester (18 mg, 0.07 mmol, 1.0 eq) was added and the resulting mixture stirred at room temperature for 20 mins. The reaction mixture was then purified by prep HPLC to give 2 fractions; 21, white solid, 18 mg (16%) and 22, white solid, 41 mg (41%). 21 LC/MS rt 1.28 min m/z (1499.8) M+H. 22 LC/MS rt 1.07 min m/z (1367.1) M−H.

### k) 4-((2R,5R)-38-(2,5-dioxo-2,5-dihydro-1H-pyrrol-1-yl)-5-isopropyl-2-methyl-4,7,34,36-tetraoxo-10,13,16,19,22,25,28,31-octaoxa-3,6,35-triazaoctatriacontanamido)benzyl (2S,11S,11aS)-8-(3-(((2S,11S,11aS)-2,11-dihydroxy-10-(((4-((10R,13R)-10-isopropyl-13-methyl-8,11-dioxo-2, 5-dioxa-9,12-diazatetradecan-14-amido)benzyl)oxy)carbonyl)-7-methoxy-5-oxo-2, 3,5,10,11,11a-hexahydro-1H-benzo[e]pyrrolo[1, 2-a][1,4]diazepin-8-yl)oxy)propoxy)-2,11-dihydroxy-7-methoxy-5-oxo-2,3,11,11a-tetrahydro-1H-benzo[e]pyrrolo[1,2-a][1,4]diazepine-10(5H)-carboxylate 23

Sodium hydrogen carbonate (3 mg, 0.036 mmol, 1.2 eq) was dissolved in water (0.5 mL) and added to a solution of 22 (41 mg, 0.03 mmol, 1.0 eq) in THF (0.5 mL). Mal-PEG₈-COOH NHS ester (23 mg, 0.033 mmol, 1.1 eq) was added and the resulting mixture stirred at room temperature for 30 mins. The reaction mixture was then purified by prep HPLC to leave 23 as a white solid, 16 mg (28%). LC/MS rt 1.31 min m/z (1944.45) M+H.

### Example 3—Conjugation

Conj-HER-23

Site-specific tratuzumab (30 mg) was loaded onto solid support and reduced, reoxidised, conjugated to compound 23, purified, released from the resin and formulated onto 25 mM Histidine, 200 mM Sucrose, Tween-20 0.02%, pH 6.0 according to patent US2014/038041A1.

UHPLC analysis on a Shimadzu Prominence system using a Thermo Scientific MAbPac 50 mm×2.1 mm mm column eluting with a gradient of water and acetonitrile on a reduced sample of Conjugate at 214 nm and 330 nm (compound 23 specific) shows unconjugated light chains and a mixture of unconjugated heavy chains and heavy chains attached to a single molecule of compound 23, consistent with a drug-per-antibody ratio (DAR) of 1.9 molecules of compound 23 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ADC at 280 nm shows a monomer purity of greater than 97%. UHPLC SEC analysis gives a concentration of final ADC at 1.7 mg/mL in 10.5 mL, obtained mass of ADC is 17.9 mg (60% yield).

Conj-HER-23*

A 10 mM solution of (TCEP) in phosphate-buffered saline pH 7.4 (PBS) was added (2.1 molar equivalent/antibody, 210 nanomoles, 21 μL) to a 7.5 mL solution of tratuzumab (15 mg, 100 nanomoles) in reduction buffer containing PBS and 1 mM ethylenediaminetetraacetic acid (EDTA) and a final antibody concentration of 2.0 mg/mL. The reduction mixture was allowed to react at +37° C. for 2 hours in an orbital shaker with gentle (60 rpm) shaking. The reduced antibody solution was allowed to cool to room temperature and compound 23 was added as a DMSO solution (10 molar equivalent/antibody, 1.0 micromoles, in 0.75 mL DMSO) to 7.5 mL of this reduced antibody solution (15 mg, 100 nanomoles) for a 10% (v/v) final DMSO concentration and a final antibody concentration of ˜2 mg/mL. The solution was mixed for 1 hour at room temperature. UHPLC analysis showed drug-per-antibody ratio (DAR) was too low therefore conjugation mixture was purified via spin filter centrifugation into PBS+1 mM EDTA using a 15 mL Amicon Ultracell 50 KDa MWCO spin filter, and more TCEP (0.8 molar equivalent/antibody, 80 nanomoles, 8 μL) was added to 5 mL of this buffer exchanged conjugation mixture (15 mg antibody, 100 nanomoles) at ˜3 mg/mL antibody concentration. The new reduction mixture was allowed to react at +37° C. for 1.75 hours in an orbital shaker with gentle (60 rpm) shaking. The reduced antibody solution was allowed to cool to room temperature and compound 23 was added as a DMSO solution (3 molar equivalent/antibody, 0.3 micromoles, in 0.5 mL DMSO) to 5 mL of this reduced antibody solution (15 mg, 100 nanomoles) for a 10% (v/v) final DMSO concentration and a final antibody concentration of ˜3 mg/mL. The solution was mixed for 16 hours at room temperature, then the conjugation was quenched by addition of N-acetyl cysteine (1.5 micromoles, 15 μL at 100 mM), then purified via spin filter centrifugation into 25 mM Histidine 205 mM Sucrose pH 6.0 buffer using a 15 mL Amicon Ultracell 50 KDa MWCO spin filter, sterile-filtered and analysed. Conj-Her-23* was then buffer exchanged into PBS via spin filter centrifugation, sterile filtered and analysed.

UHPLC analysis on a Shimadzu Prominence system using a Thermo Scientific MAbPac 50 mm×2.1 mm column eluting with a gradient of water and acetonitrile on a reduced sample of Conj-Her-23* at 214 nm shows a mixture of unconjugated light chains, light chains attached to a single molecule of compound 23, unconjugated heavy chains and heavy chains attached to up to three molecules of compound 23 consistent with a drug-per-antibody ratio (DAR) of 3.87 molecules of compound 23 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of Conj-Her-23* at 280 nm shows a monomer purity of 99%. Reduced SDS-PAGE analysis gives a concentration of final Conj-Her-23* at 1.16 mg/mL in 4.4 mL, obtained mass of Conj-Her-23* is 5.1 mg (34% yield).

Conj-Her-23**

A 10 mM solution of (TCEP) in phosphate-buffered saline pH 7.4 (PBS) was added (10 molar equivalent/antibody, 1 micromole, 100 μL) to a 7.5 mL solution of tratuzumab (15 mg, 100 nanomoles) in reduction buffer containing PBS and 1 mM ethylenediaminetetraacetic acid (EDTA) and a final antibody concentration of 2.0 mg/mL. The reduction mixture was allowed to react at +37° C. for 3 hours (or until full reduction is observed by UHPLC) in an orbital shaker with gentle (60 rpm) shaking. The reduced antibody solution was allowed to cool to room temperature and diluted with 6 mL more PBS and 1 mM EDTA. Compound 23 was added as a DMSO solution (15 molar equivalent/antibody, 1.5 micromoles, in 1.5 mL DMSO) to 13.5 mL of this reduced antibody solution (15 mg, 100 nanomoles) for a 10% (v/v) final DMSO concentration and a final antibody concentration of 1.0 mg/mL. The solution was mixed for 16 hours at room temperature, then the conjugation was quenched by addition of N-acetyl cysteine (7.5 micromoles, 75 μL at 100 mM), then purified via spin filter centrifugation into 25 mM Histidine 205 mM Sucrose pH 6.0 buffer using a 15 mL Amicon Ultracell 50 KDa MWCO spin filter, sterile-filtered and analysed. Conj-Her-23** was then buffer exchanged into PBS via spin filter centrifugation, sterile filtered and analysed.

UHPLC analysis on a Shimadzu Prominence system using a Thermo Scientific MAbPac 50 mm×2.1 mm column eluting with a gradient of water and acetonitrile on a reduced sample of Conj-Her-23** at 214 nm shows a mixture of unconjugated light chains, light chains attached to a single molecule of compound 23, unconjugated heavy chains and heavy chains attached to up to three molecules of compound 23, consistent with a drug-per-antibody ratio (DAR) of 7.60 molecules of compound 23 per antibody.

UHPLC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of Conj-Her-23** at 280 nm shows a monomer purity of 95%. Reduced SDS-PAGE analysis gives a concentration of final Conj-Her-23** at 0.2 mg/mL in 6 mL, obtained mass of Conj-Her-23** is 1.2 mg (8% yield).

### Example 4—In Vivo Assay

Conjugate Tested: Conj-Her-23

Female CB.17 SCID mice, aged ten weeks, were injected with 0.1 ml of 1×10⁷ NCl—N87 cells in 50% Matrigel subcutaneously in the right flank. When tumours reached an average size of 100-150 mm³, treatment began. Mice were weighed twice a week. Tumour size was measured twice a week. Animals were monitored individually. The endpoint of the experiment was a tumour volume of 800 mm³ or 83 days, whichever came first.

Groups of 10 xenografted mice were injected i.v. with 0.2 ml per 20 g of body weight of antibody drug conjugate (ADC) in phosphate buffered saline (vehicle) or with 0.2 ml per 20 g of body weight of vehicle alone. The concentration of ADC was adjusted to give 0.6 or 6 mg ADC/kg body weight in a single dose.

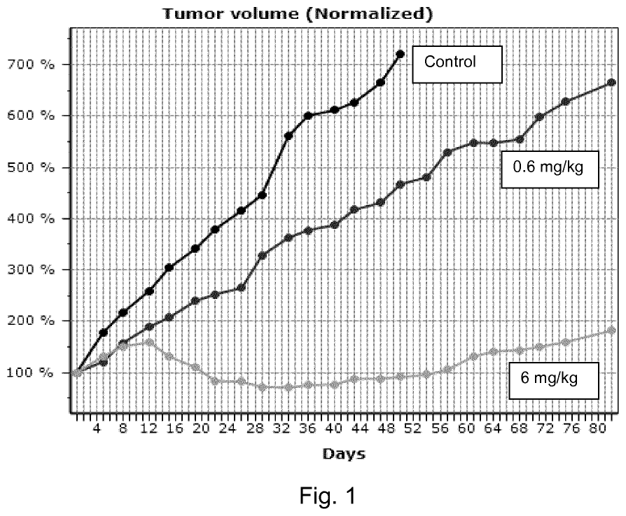

The change in normalised tumour volume over time is shown in FIG. 1.

Endpoint and Tumor Growth Delay (TGD) Analysis

Tumors were measured using calipers twice per week, and each animal was euthanized when its tumor reached the endpoint volume of 800 mm³ or at the end of the study (Day 82), whichever came first. Animals that exited the study for tumor volume endpoint were documented as euthanized for tumor progression (TP), with the date of euthanasia. The time to endpoint (TTE) for analysis was calculated for each mouse by the following equation:

$$TTE = \frac{{log}_{10} ( endpoint volume ) - b}{m}$$

where TTE is expressed in days, endpoint volume is expressed in mm³, b is the intercept, and m is the slope of the line obtained by linear regression of a log-transformed tumor growth data set. The data set consisted of the first observation that exceeded the endpoint volume used in analysis and the three consecutive observations that immediately preceded the attainment of this endpoint volume. The calculated TTE is usually less than the TP date, the day on which the animal was euthanized for tumor size. Animals with tumors that did not reach the endpoint volume were assigned a TTE value equal to the last day of the study (Day 82). In instances in which the log-transformed calculated TTE preceded the day prior to reaching endpoint or exceeded the day of reaching tumor volume endpoint, a linear interpolation was performed to approximate the TTE. Any animal classified as having died from NTR (non-treatment-related) causes due to accident (NTRa) or due to unknown etiology (NTRu) were excluded from TTE calculations (and all further analyses). Animals classified as TR (treatment-related) deaths or NTRm (non-treatment-related death due to metastasis) were assigned a TTE value equal to the day of death. Treatment outcome was evaluated from tumor growth delay (TGD), which is defined as the increase in the median time to endpoint (TTE) in a treatment group compared to the control group:

TGD=T−C,

expressed in days, or as a percentage of the median TTE of the control group:

$$\% TGD = \frac{T - C}{C} \times 100$$

where:
T=median TTE for a treatment group, and
C=median TTE for the designated control group.

Tumour Growth Inhibition

Tumor growth inhibition (TGI) analysis evaluates the difference in median tumor volumes (MTVs) of treated and control mice. For this study, the endpoint for determining TGI was Day 33, which was the last day that all evaluable control mice remained in the study. The MTV (n), the median tumor volume for the number of animals, n, on the day of TGI analysis, was determined for each group. Percent tumor growth inhibition (% TGI) was defined as the difference between the MTV of the designated control group and the MTV of the drug-treated group, expressed as a percentage of the MTV of the control group:

$$\% TGI = ( \frac{{MTV}_{control} - M T {V}_{drug - treated}}{{MTV}_{control}} ) \times 100 = [ 1 - ( MT {V}_{drug - treated} / {MTV}_{control} ) ] \times 100$$

The data set for TGI analysis included all animals in a group, except those that died due to treatment-related (TR) or non-treatment-related (NTR) causes prior to the day of TGI analysis.

MTV and Criteria for Regression Responses

Treatment efficacy may be determined from the tumor volumes of animals remaining in the study on the last day. The MTV (n) was defined as the median tumor volume on the last day of the study in the number of animals remaining (n) whose tumors had not attained the endpoint volume. Treatment efficacy may also be determined from the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study. Animals were scored only once during the study for a PR or CR event and only as CR if both PR and CR criteria were satisfied. An animal with a CR response at the termination of a study was additionally classified as a tumor-free survivor (TFS). Animals were monitored for regression responses.

Toxicity

Animals were weighed daily on Days 1-5, then twice per week until the completion of the study. The mice were observed frequently for overt signs of any adverse, treatment-related (TR) side effects, and clinical signs were recorded when observed. Individual body weight was monitored as per protocol, and any animal with weight loss exceeding 30% for one measurement or exceeding 25% for three consecutive measurements was euthanized as a TR death. Group mean body weight loss was also monitored according to CR Discovery Services protocol. Acceptable toxicity was defined as a group mean body weight (BW) loss of less than 20% during the study and no more than 10% TR deaths. Dosing was suspended in any group where mean weight loss exceeded acceptable limits. If group mean body weight recovered to acceptable levels, then dosing was modified to lower levels and/or reduced frequency then resumed. Deaths were classified as TR if it was attributable to treatment side effects as evidenced by clinical signs and/or necropsy. A TR classification was also assigned to deaths by unknown causes during the dosing period or within 14 days of the last dose. A death was classified as non-treatment-related (NTR) if there was no evidence that death was related to treatment side effects. NTR deaths are further categorized as follows: NTRa describes deaths due to accidents or human error; NTRm is assigned to deaths thought to result from tumor dissemination by invasion and/or metastasis based on necropsy results; NTRu describes deaths of unknown causes that lack available evidence of death related to metastasis, tumor progression, accident or human error. It should be noted that treatment side effects cannot be excluded from deaths classified as NTRu.

Statistical and Graphical Analyses

GraphPad Prism 8.0 for Windows was used for all statistical analysis and graphical presentations. Study groups experiencing toxicity beyond acceptable limits (>20% group mean body weight loss or greater than 10% treatment-related deaths) or having fewer than five evaluable observations, were not included in the statistical analysis. The logrank test was employed to assess the significance of the difference between the overall survival experiences of two groups. The logrank test analyzes the individual TTEs for all animals in a group, except those lost to the study due to NTR death. Statistical analyses of the differences between Day 33 median tumor volumes (MTVs) of control and treated groups were accomplished using the Mann-Whitney U-test. For statistical analyses, two-tailed tests were conducted at significance level P=0.05. Prism summarizes test results as not significant (ns) at P>0.05, significant (symbolized by “*”) at 0.01<P≤0.05, very significant (“**”) at 0.001<P≤0.01, and extremely significant (“***”) at P≤0.001. Because tests of statistical significance do not provide an estimate of the magnitude of the difference between groups, all levels of significance were described as either significant or not significant within the text of this report. A scatter plot was constructed to show TTE values for individual mice, by group. Tumor growth curves show group median, mean and individual tumor volumes as a function of time, with error bars (when present) indicating one standard error of the mean (SEM). When an animal exited the study due to tumor size, the final tumor volume recorded for the animal was included with the data used to calculate the mean volume at subsequent time points. Tumor growth curves were truncated when tumors in more than 50% of the assessable animals in the group grew to the endpoint volume and excluded the data for animals whose deaths were assessed as NTR. Kaplan-Meier plots show the percentage of animals in each group remaining in the study versus time. The Kaplan-Meier plot and logrank test share the same TTE data sets. Box and whisker plots were constructed to show the Day 33 tumor volume data by group, with the “box” representing the 25th and 75^{th} percentile of observations, the “line” representing the median of observations, and the “whiskers” representing the extreme observations. Group body weight changes during the study were plotted as percent mean change from Day 1. Body weight plots were truncated after 50% of the assessable animals in a group had exited the study and excluded the data for animals whose deaths were assessed as NTR.

| | | | Median | | % | MTV (n), |
|---|---|---|---|---|---|---|
| | | n | TTE | T—C | TGD | Day 82 |
| Vehicle | — | 10 | 45.7 | — | — | — |
| Conj-Her-23 | 0.6 mg/kg | 10 | 74.0 | 28.2 | 62 | 507 (2) |
| Conj-Her-23 | 6 mg/kg | 10 | 82.0 | 36.2 | 79 | 63 (9) |

| | | | | BW | | | | |
|---|---|---|---|---|---|---|---|---|
| | PR | CR | TFS | Nadir | | TR | NTRm | NTR |
| Vehicle | 0 | 0 | 0 | −0.5% | (3) | 0 | 0 | 0 |
| 0.6 mg/kg | 0 | 0 | 0 | −0.8* | (71) | 0 | 0 | 0 |

| | | | | | | | |
|---|---|---|---|---|---|---|---|
| 6 mg/kg | 5 | 1 | 1 | — | 0 | 0 | 0 |

All documents and other references mentioned above are herein incorporated by reference.

### EMBODIMENTS OF INVENTION

1. A compound of formula I:

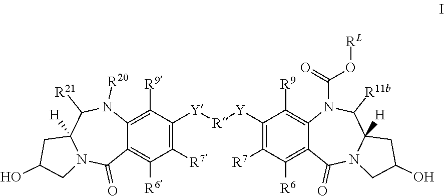

and salts and solvates thereof, wherein:
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
R^{11b} is selected from OH, OR^{A}, where R^{A} is C₁₋₄ alkyl; and
R^{L} is a linker for connection to a cell binding agent, which is selected from: (iiia):

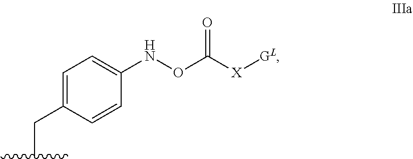

wherein

### Q is:

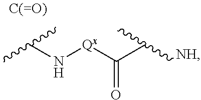

where Q^{X} is such that Q is an amino-acid residue, a dipeptide residue or a tripeptide residue;

### X is:

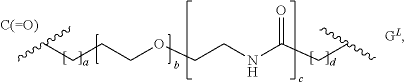

where a=0 to 5, b=0 to 16, c=0 or 1, d=0 to 5;
G^{L} is a linker for connecting to a Ligand Unit; and
(iiib):

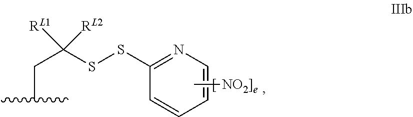

where R^{L1} and R^{L2} are independently selected from H and methyl, or together with the carbon atom to which they are bound form a cyclopropylene or cyclobutylene group;
and e is 0 or 1;
either:
(a) R²⁰ is H and R²¹ is H;
(b) R²⁰ is H and R²¹ is ═O; or
(c) R²¹ is OH or OR^{A}, where R^{A} is C₁₋₄ alkyl and R²⁰ is selected from:

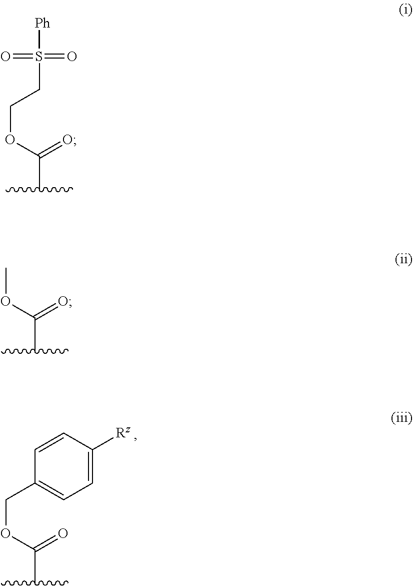

where R^{Z} is selected from:

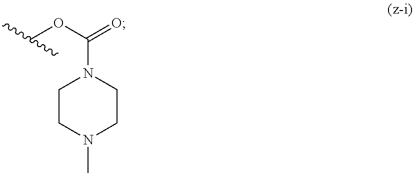 [SMILES: *OC(=O)N1CCN(C)CC1]

(z-ii) OC(═O)CH₃;
(z-iii) NO₂;
(z-iv) OMe;
(z-v) glucoronide;
(z-vi) NH—C(═O)—X₁—NHC(═O)X₂—NH—C(═O)—R^{ZC}, where —C(═O)—X₁—NH— and —C(═O)—X₂—NH— represent natural amino acid residues and R^{ZC} is selected from Me, OMe, CH₂CH₂OMe, and (CH₂CH₂O)₂Me.
   2. A compound according to statement 1, wherein both Y and Y′ are O.
   3. A compound according to either statement 1 or statement 2, wherein R″ is C₃₋₇ alkylene.
   4. A compound according to either statement 1 or statement 2, wherein R″ is a group of formula:

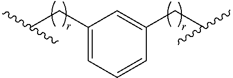

where r is 1 or 2.
5. A compound according to any one of statements 1 to 4, wherein R⁹ is H.
6. A compound according to any one of statements 1 to 5, wherein R⁶ is H.
7. A compound according to any one of statements 1 to 6, wherein R⁷ is selected from H, OH and OR.
8. A compound according to statement 7, wherein R⁷ is a C₁₋₄ alkyloxy group.
9. A compound according to any one of statements 1 to 8, wherein R^{6′} is the same group as R⁶, R^{7′} is the same group as R⁷, R^{9′} is the same group as R⁹ and Y′ is the same group as Y.
10. The compound according to any one of statements 1 to 9, wherein R²¹ is OH or OR^{A} and R²⁹ is selected from:

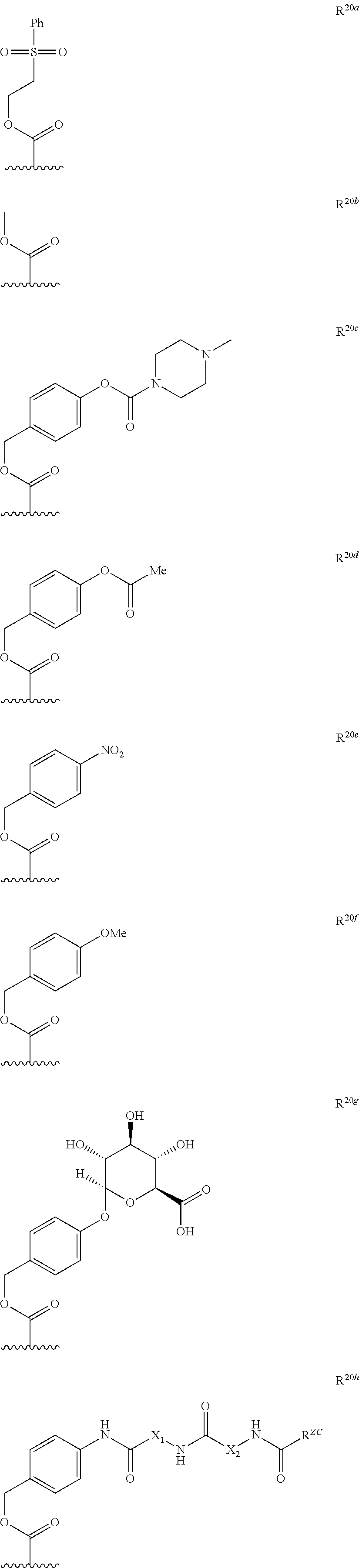

11. The compound according to any one of statements 1 to 10, wherein —C(═O)—X₁—NHC(═O)X₂—NH—, is selected from: -Phe-Lys-, -Val-Ala-, -Val-Lys-, -Ala-Lys-, and -Val-Cit-.
12. The compound according to statement 11, wherein —C(═O)—X₁—NHC(═O)X₂—NH—, is selected from: -Phe-Lys-, and -Val-Ala-.
13. The compound according to any one of statements 10 to 12 wherein R^{ZC} is selected from CH₂CH₂OMe, and (CH₂CH₂O)₂Me.
14. The compound according to statement 13 wherein R^{ZC} is (CH₂CH₂O)₂Me.
15. A compound according to statement 1, which is of formula Ia, Ib or Ic:

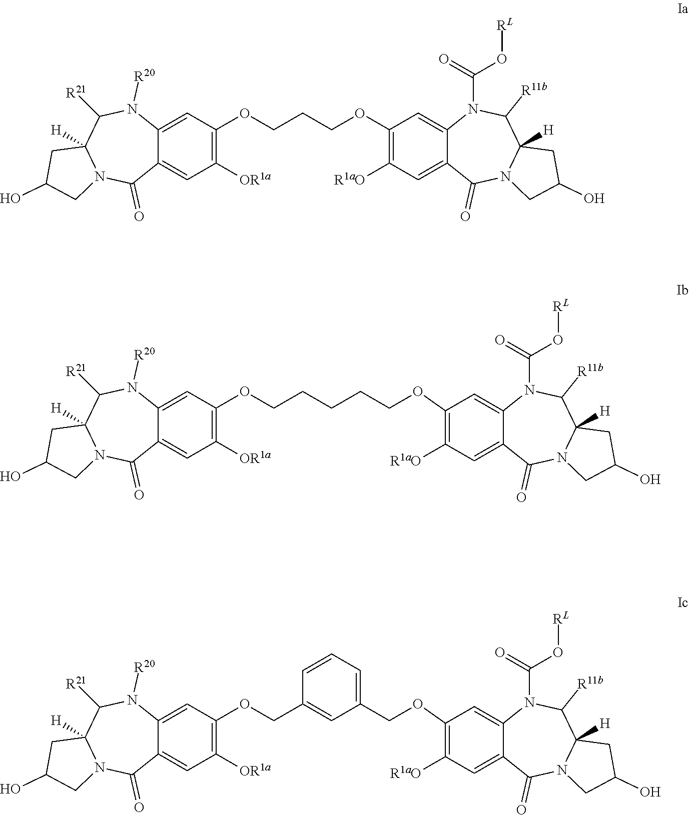

where R^{1a} is selected from methyl and benzyl;
R^{L} and R^{11b} are as defined in statement 1.
16. A compound according to any one of statements 1 to 15, wherein R^{11b} is OH.
17. A compound according to any one of statements 1 to 15, wherein R^{11b} is OR^{A}, where R^{A} is C₁₋₄ alkyl.
18. A compound according to statement 17, wherein R^{A} is methyl.
19. A compound according to any one of statements 1 to 18, wherein R^{L} is of formula IIIa, and Q is an amino acid residue selected from Phe, Lys, Val, Ala, Cit, Leu, Ile, Arg, and Trp.
20. A compound according to any one of statements 1 to 18, wherein R^{L} is of formula IIIa, and Q is a dipeptide residue selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{NH},
^{CO}-Phe-Cit-^{NH},
^{CO}-Leu-Cit-^{NH},
^{CO}-Ile-Cit-^{NH},
^{CO}-Phe-Arg-^{NH}, and
^{CO}-Trp-Cit-^{NH}.
   21. A compound according to statement 20, wherein Q is selected from ^{CO}-Phe-Lys-^{NH}, ^{CO}-Val-Cit-^{NH} and ^{CO}-Val-Ala-^{NH}.
   22. A compound according to any one of statements 1 to 18, wherein R^{L} is of formula IIIa, and Q is a tripeptide residue.
   23. A compound according to any one of statements 1 to 22, wherein R^{L} is of formula IIIa and a is 0 to 3.
   24. A compound according to statement 23, wherein a is 0.
   25. A compound according to any one of statements 1 to 24, wherein R^{L} is of formula IIIa and b is 0 to 12.
   26. A compound according to statement 25, wherein b is 0 to 8.
   27. A compound according to any one of statements 1 to 26, wherein R^{L} is of formula IIIa and d is 0 to 3.
   28. A compound according to statement 27, wherein d is 2.
   29. A compound according to any one of statements 1 to 22, wherein R^{L} is of formula IIIa and, a is 0, c is 1 and d is 2, and b is from 0 to 8.
   30. A compound according to statement 29, wherein b is 0, 4 or 8.
   31. A compound according to any one of statements 1 to 30, wherein R^{L} is of formula IIIa and G^{L} is selected from:

| | |
|---|---|
| 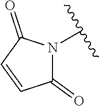 [SMILES: *N1C(=O)C=CC1=O] | (G^{L1-1}) |
| 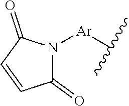 | (G^{L1-2}) |
| 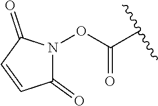 [SMILES: *C(=O)ON1C(=O)C=CC1=O] | (G^{L2}) |
| 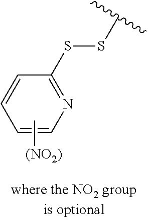 | (G^{L3-1}) |
| 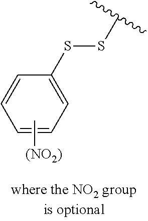 | (G^{L3-2}) |
| 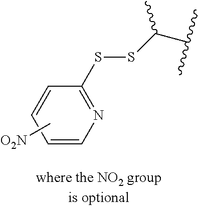 | (G^{L3-3}) |
| 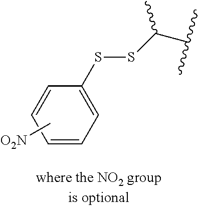 | (G^{L3-4}) |
| 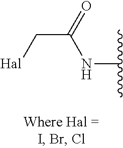 | (G^{L4}) |
| 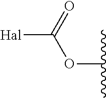 | (G^{L5}) |
| 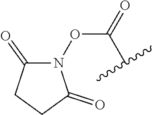 [SMILES: *C(=O)ON1C(=O)CCC1=O] | (G^{L6}) |
| 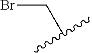 [SMILES: *CBr] | (G^{L7}) |
| 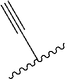 [SMILES: *C#C] | (G^{L8}) |
| 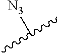 [SMILES: *N=[N+]=[N-]] | (G^{L9}) |

where Ar represents a C₅₋₆ arylene group.
32. A compound according to statement 31, wherein Ar is a phenylene group.
33. A compound according to either statement 31 or statement 32, wherein G^{L} is selected from G^{L1-1} and G^{L1-2}.
34. A compound according to statement 33, wherein G^{L} is G^{L1-1}.
35. A compound according to any one of statements 1 to 18, wherein R^{L} is of formula IIIb, and both R^{L1} and R^{L2} are H.
36. A compound according to any one of statements 1 to 18, wherein R^{L} is of formula IIIb, R^{L1} is H and R^{L2} is methyl.
37. A compound according to any one of statements 1 to 18, wherein R^{L} is of formula IIIb, and both R^{L1} and R^{L2} are methyl.
38. A compound according to any one of statements 1 to 18, wherein R^{L} is of formula IIIb, and, R^{L1} and R^{L2} together with the carbon atom to which they are bound form a cyclopropylene group.
39. A compound according to any one of statements 1 to 18, wherein R^{L} is of formula IIIb, and, R^{L1} and R^{L2} together with the carbon atom to which they are bound form a cyclobutylene group.
40. A compound according to any one of statements 1 to 18 and 35 to 39, wherein R^{L} is of formula IIIb, and e is 0.
41. A compound according to any one of statements 1 to 18 and 35 to 39, wherein R^{L} is of formula IIIb, and e is 1.
42. A compound according to statement 41, wherein the nitro group is in the para position.
43. A compound according to statement 1, wherein the compound is of formula Id:

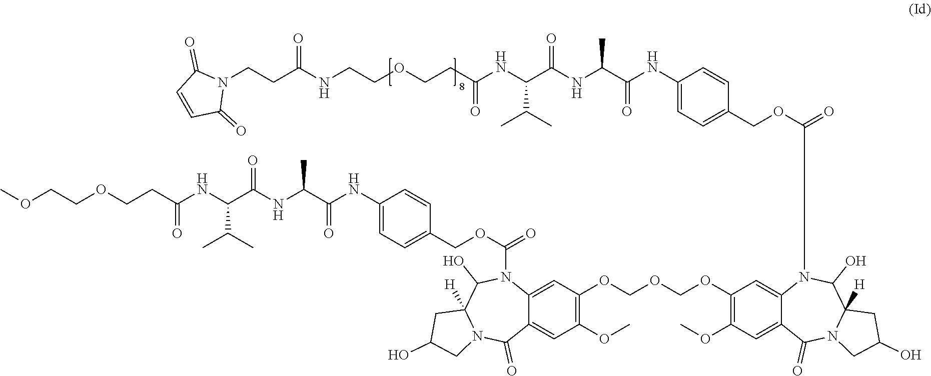

where Q is selected from:

### (a) —CH₂—;

(b) —C₃H₆—; and

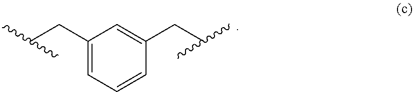 [SMILES: *Cc1cccc(C*)c1]

44. A conjugate of formula II:

L-(D^{L})ₚ (I)

wherein L is a Ligand unit, D^{L} is a Drug Linker unit of formula I′:

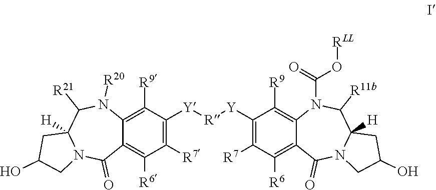

wherein R⁶, R⁷, R⁹, R^{11b}, Y, R″, Y′, R^{6′}, R⁷, R^{9′}, R²⁰ and R²¹, are as defined in any one of statements 1 to 18;
R^{LL} is a linker for connection to a cell binding agent, which is selected from:
(iiia):

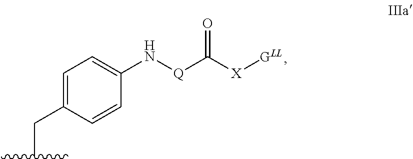

where Q and X are as defined in any one of statements 1 and 19 to 21 and G^{LL} is a linker connected to a Ligand Unit; and
(iiib):

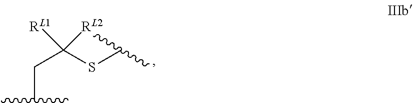

where R^{L1} and R^{L2} are as defined in any one of statements 1 and 35 to 39;
wherein p is an integer of from 1 to 20.
45. A conjugate according to statement 44, wherein G^{LL} is selected from:

| | |
|---|---|
| 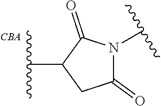 [SMILES: *C1CC(=O)N(*)C1=O] | (G^{LL1-1}) |
| 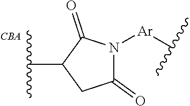 | (G^{LL1-2}) |
| 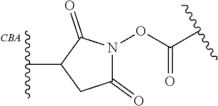 [SMILES: *C(=O)ON1C(=O)CC(*)C1=O] | (G^{LL2}) |
| 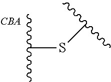 [SMILES: *S*] | (G^{LL3-1}) |
| 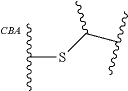 [SMILES: *SC(*)C] | (G^{LL3-2}) |
| 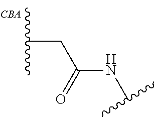 [SMILES: *CC(=O)N*] | (G^{LL4}) |
| 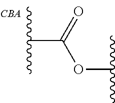 [SMILES: *OC(*)=O] | (G^{LL5}) |
| 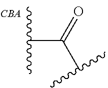 [SMILES: *C(*)=O] | (G^{LL6}) |
| 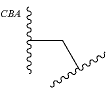 [SMILES: *C*] | (G^{LL7}) |
| 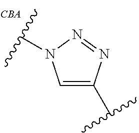 [SMILES: *c1cn(*)nn1] | (G^{LL8-1}) |
| 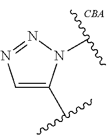 [SMILES: *c1cnnn1*] | (G^{LL8-2}) |
| 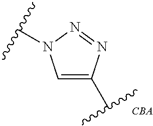 [SMILES: *c1cn(*)nn1] | (G^{LL9-1}) |
| 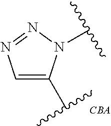 [SMILES: *c1cnnn1*] | (G^{LL9-2}) |

where Ar represents a C₅₋₆ arylene group.
46. A conjugate according to statement 45, wherein Ar is a phenylene group.
47. A conjugate according to either statement 45 or statement 46, wherein G^{LL} is selected from G^{LL1-1} and GL^{L1-2}.
48. A conjugate according to statement 47, wherein G^{LL} is GL^{L1-1}.
49. A conjugate according to statement 44, wherein D^{L} is of formula (Id′):

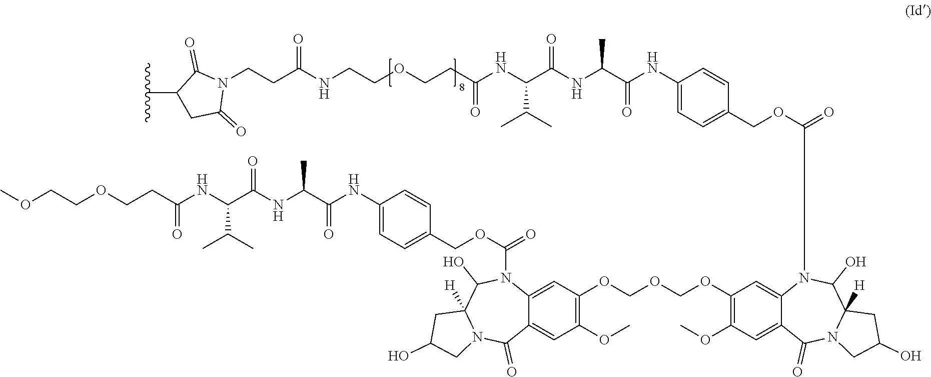

where Q is selected from:

### (a) —CH₂—;

(b) —C₃H₆—; and

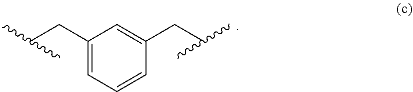 [SMILES: *Cc1cccc(C*)c1]

50. A conjugate according to any one of statements 44 to 49, wherein the Ligand Unit is an antibody or an active fragment thereof.
51. The conjugate according to statement 50, wherein the antibody or antibody fragment is an antibody or antibody fragment for a tumour-associated antigen.
52. The conjugate according to statement 51 wherein the antibody or antibody fragment is an antibody which binds to one or more tumor-associated antigens or cell-surface receptors selected from (1)-(89):

### (1) BMPR1B;

### (2) E16;

### (3) STEAP1;

### (4) 0772P;

### (5) MPF;

### (6) Napi3b;

### (7) Sema 5b;

(8) PSCA hlg;

### (9) ETBR;

### (10) MSG783;

### (11) STEAP2;

### (12) TrpM4;

### (13) CRI PTO;

### (14) CD21;

### (15) CD79b;

### (16) FcRH2;

### (17) HER2;

### (18) NCA;

### (19) MDP;

### (20) IL20R-alpha;

### (21) Brevican;

### (22) EphB2R;

### (23) ASLG659;

### (24) PSCA;

### (25) GEDA;

### (26) BAFF-R;

### (27) CD22;

### (28) CD79a;

### (29) CXCR5;

### (30) HLA-DOB;

### (31) P2X5;

### (32) CD72;

### (33) LY64;

### (34) FcRH1;

### (35) IRTA2;

### (36) TENB2;

### (37) PSMA-FOLH1;

### (38) SST;

### (38.1) SSTR2;

### (38.2) SSTR5;

### (38.3) SSTR1;

### (38.4) SSTR3;

### (38.5) SSTR4;

### (39) ITGAV;

### (40) ITGB6;

### (41) CEACAM5;

### (42) MET;

### (43) MUC1;

### (44) CA9;

### (45) EGFRvIII;

### (46) CD33;

### (47) CD19;

### (48) IL2RA;

### (49) AXL;

### (50) CD30-TNFRSF8;

### (51) BCMA-TNFRSF17;

### (52) CT Ags-CTA;

### (53) CD174 (Lewis Y)-FUT3;

### (54) CLEC14A;

### (55) GRP78-HSPA5;

### (56) CD70;

(57) Stem Cell specific antigens;

### (58) ASG-5;

### (59) ENPP3;

### (60) PRR4;

### (61) GCC-GUCY2C;

### (62) Liv-1-SLC39A6;

### (63) 5T4;

### (64) CD56-NCMA1;

### (65) CanAg;

### (66) FOLR1;

### (67) GPNMB;

### (68) TIM-1-HAVCR1;

(69) RG-1/Prostate tumor target Mindin-Mindin/RG-1;

### (70) B7-H4-VTCN1;

### (71) PTK7;

### (72) CD37;

### (73) CD138-SDC1;

### (74) CD74;

### (75) Claudins-CLs;

### (76) EGFR;

### (77) Her3;

### (78) RON-MST1R;

### (79) EPHA2;

### (80) CD20-MS4A1;

### (81) Tenascin C-TNC;

### (82) FAP;

### (83) DKK-1;

### (84) CD52;

### (85) CS1-SLAMF7;

### (86) Endoglin-ENG;

### (87) Annexin A1-ANXA1;

### (88) V-CAM (CD106)-VCAM1;

### (89) ASCT2 (SLC1A5).

53. The conjugate of any one of statements 50 to 52 wherein the antibody or antibody fragment is a cysteine-engineered antibody.
54. The conjugate according to any one of statements 44 to 53 wherein p is an integer from 1 to 8.
55. The conjugate according to statement 54, wherein p is 1, 2, 3, or 4.
56. A composition comprising a mixture of conjugates according to any one of statements 44 to 55, wherein the average p in the mixture of conjugate compounds is about 1 to about 8.
57. The conjugate according to any one of statements 44 to 55, for use in therapy.
58. A pharmaceutical composition comprising the conjugate of any one of statements 44 to 55, and a pharmaceutically acceptable diluent, carrier or excipient.
59. The conjugate according to any one of statements 44 to 55 or the pharmaceutical composition according to statement 58, for use in the treatment of a proliferative disease in a subject.
60. The conjugate for use according to statement 61, wherein the disease treated is cancer.
61. Use of a conjugate according to any one of statements 44 to 55 or a pharmaceutical composition according to statement 58 in a method of medical treatment.
62. A method of medical treatment comprising administering to a patient the pharmaceutical composition of statement 58.
63. The method of statement 62 wherein the method of medical treatment is for treating cancer.
64. The method of statement 63, wherein the patient is administered a chemotherapeutic agent, in combination with the conjugate.
65. Use of a conjugate according to any one of statements 44 to 55 in a method of manufacture of a medicament for the treatment of a proliferative disease.
66. A method of treating a mammal having a proliferative disease, comprising administering an effective amount of a conjugate according to any one of statements 44 to 55 or a pharmaceutical composition according to statement 58.
67. A compound of Formula IV:

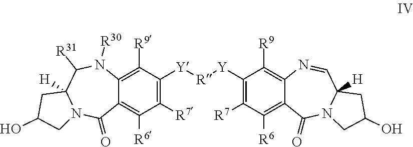

wherein R⁶, R⁷, R⁹, Y, R″, Y′, R^{6′}, R⁷ and R^{9′}, are as defined in any one of statements 1 to 18;
either:
(a) R³⁹ is H and R³¹ is H;
(b) R³⁹ is H and R³¹ is ═O; or
(c) R³⁹ and R³¹ form a double bond between the N and C atoms to which they are attached.

1. A compound of formula I: 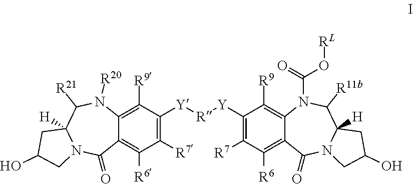
and salts and solvates thereof, wherein:
R⁶ and R⁹ are independently selected from H, R, OH, OR, SH, SR, NH₂, NHR, NRR′, nitro, Me₃Sn and halo;
where R and R′ are independently selected from C₁₋₁₂ alkyl, C₃₋₂₀ heterocyclyl and C₅₋₂₀ aryl groups;
R⁷ is selected from H, R, OH, OR, SH, SR, NH₂, NHR, NRR′, nitro, Me₃Sn and halo;
R″ is a C₃₋₁₂ alkylene group, which chain may be interrupted by one or more heteroatoms, selected from O, S, and NR^{N2} where R^{N2} is H or C₁₋₄ alkyl, and/or an aromatic ring selected from benzene or pyridine;
Y and Y′ are selected from O, S, or NH;
R^{6′}, R^{7′}, R^{9′} are selected from the same groups as R⁶, R⁷ and R⁹ respectively;
R^{11b} is selected from OH, OR^{A}, where R^{A} is C₁₋₄ alkyl; and
R^{L} is a linker for connection to a cell binding agent, which is selected from:
(iiia): 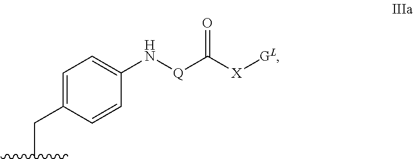
wherein
Q is: 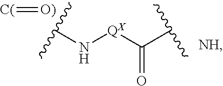
where Q^{X} is such that Q is an amino-acid residue, a dipeptide residue or a tripeptide residue;
X is: 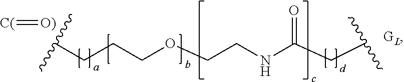
where a=0 to 5, b=0 to 16, c=0 or 1, d=0 to 5;
G^{L} is a linker for connecting to an antibody or an active fragment thereof; and
(iiib): 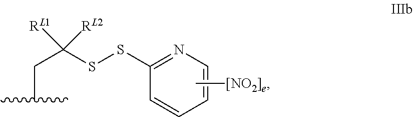
where R^{L1} and R^{L2} are independently selected from H and methyl, or together with the carbon atom to which they are bound form a cyclopropylene or cyclobutylene group;
and e is 0 or 1;
either:
(a) R²⁰ is H and R²¹ is H;
(b) R²⁰ is H and R²¹ is ═O; or
(c) R²¹ is OH or OR^{A}, where R^{A} is C₁₋₄ alkyl and R²⁰ is selected from: 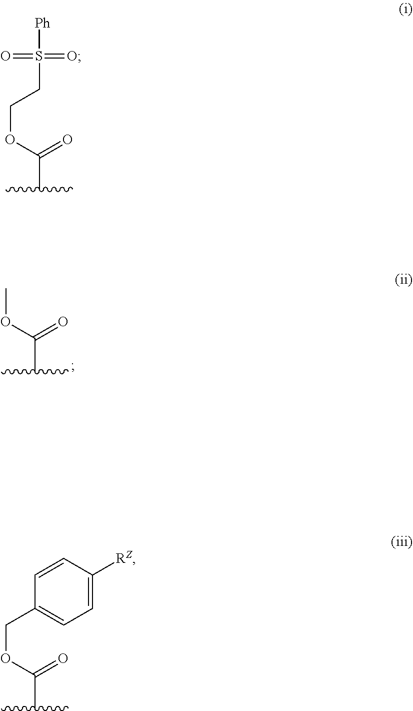
where R^{Z} is selected from: 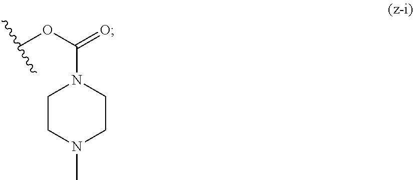 [SMILES: *OC(=O)N1CCN(C)CC1]
(z-ii) OC(═O)CH₃;
(z-iii) NO₂;
(z-iv) OMe;
(z-v) glucoronide;
(z-vi) NH—C(═O)—X₁—NHC(═O)X₂—NH—C(═O)—R^{ZC}, where —C(═O)—X₁—NH— and —C(═O)—X₂—NH— represent natural amino acid residues and R^{ZC} is selected from Me, OMe, CH₂CH₂OMe, and (CH₂CH₂O)₂Me.

2. A compound according to claim 1, wherein:
a) both Y and Y′ are O; and/or
b) R″ is C₃₋₇ alkylene or a group of formula: 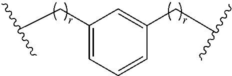
where r is 1 or 2;
c) R⁹ is H, R⁶ is H and R⁷ is a C₁₋₄ alkyloxy group.

3.-5. (canceled)

6. A compound according to claim 1, wherein R^{6′} is the same group as R⁶, R^{7′} is the same group as R⁷, R^{9′} is the same group as R⁹ and Y′ is the same group as Y.

7. The compound according to claim 1, wherein R²¹ is OH or OR^{A} and R²⁰ is 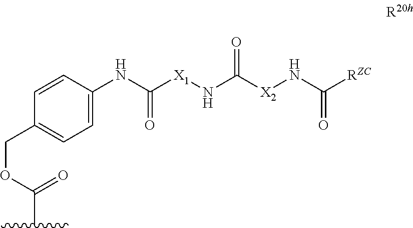
and —C(═O)—X₁—NHC(═O)X₂—NH—, is selected from: -Phe-Lys-, -Val-Ala-, -Val-Lys-, -Ala-Lys-, and -Val-Cit-.

8. The compound according to claim 7, wherein —C(═O)—X₁—NHC(═O)X₂—NH—, is selected from: -Phe-Lys-, and -Val-Ala-.

9. The compound according to either claim 7 or 8 wherein R^{ZC} is (CH₂CH₂O)₂Me.

10. A compound according to claim 1, which is of formula Ia, Ib or Ic: 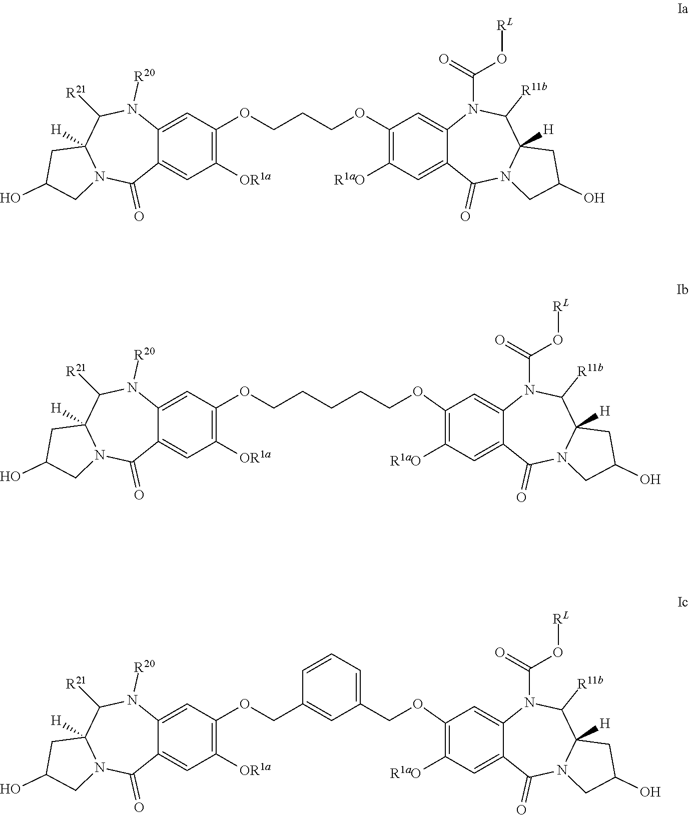
where R^{1a} is selected from methyl and benzyl;
R^{L} and R^{11b} are as defined in claim 1.

11. A compound according to claim 1, wherein R^{L} is of formula IIIa, and Q is a dipeptide residue selected from:
^{CO}-Phe-Lys-^{NH},
^{CO}-Val-Ala-^{NH},
^{CO}-Val-Lys-^{NH},
^{CO}-Ala-Lys-^{NH},
^{CO}-Val-Cit-^{NH},
^{CO}-Phe-Cit-^{NH},
^{CO}-Leu-Cit-^{NH},
^{CO}-Phe-Arg-^{NH}, and
^{CO}-Trp-Cit-^{NH}.

12. A compound according to of claim 1, wherein R^{L} is of formula IIIa and, a is 0, c is 1 and d is 2, and b is from 0 to 8.

13. A compound according to claim 12, wherein b is 0, 4 or 8.

14. A compound according to any claim 1, wherein R^{L} is of formula IIIa and G^{L} is selected from:

| | |
|---|---|
| 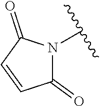 [SMILES: *N1C(=O)C=CC1=O] | (G^{L1-1}) |
| 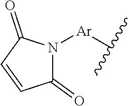 | (G^{L1-2}) |
| 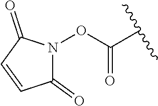 [SMILES: *C(=O)ON1C(=O)C=CC1=O] | (G^{L2}) |
| 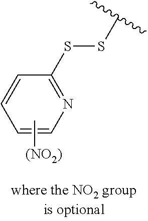 | (G^{L3-1}) |
| 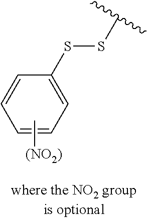 | (G^{L3-2}) |
| 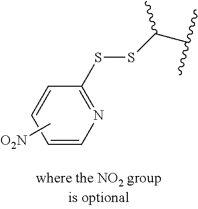 | (G^{L3-3}) |
| 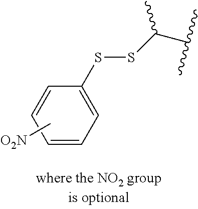 | (G^{L3-4}) |
| 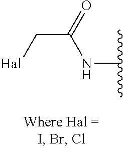 | (G^{L4}) |
| 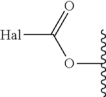 | (G^{L5}) |
| 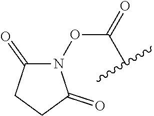 [SMILES: *C(=O)ON1C(=O)CCC1=O] | (G^{L6}) |
| 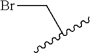 [SMILES: *CBr] | (G^{L7}) |
| 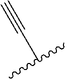 [SMILES: *C#C] | (G^{L8}) |
| 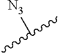 [SMILES: *N=[N+]=[N-]] | (G^{L9}) |

where Ar represents a C₅₋₆ arylene group.

15. A compound according to claim 14, wherein G^{L} is G^{L1-1}.

16. A compound according to claim 1, wherein the compound is of formula Id: 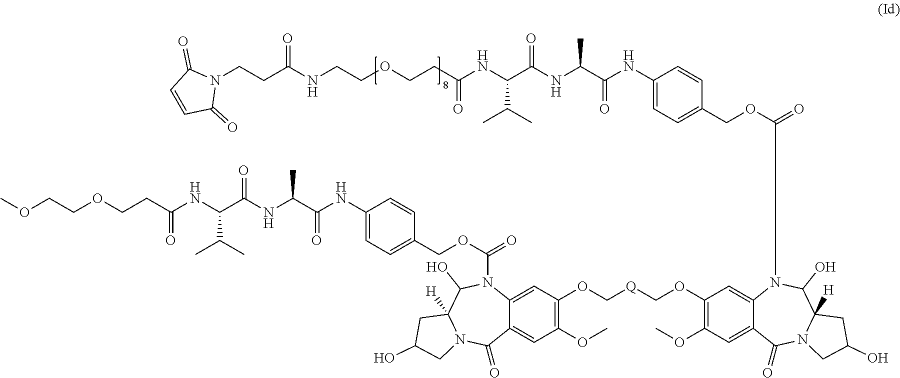
where Q is selected from:
(a) —CH₂—;
(b) —C₃H₆—; and

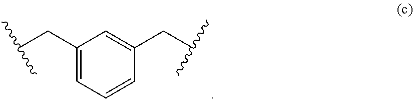 [SMILES: *Cc1cccc(C*)c1]

17. A conjugate of formula II:
L-(D^{L})ₚ (I)
wherein L is an antibody or an active fragment thereof, D^{L} is a Drug Linker unit of formula I′: 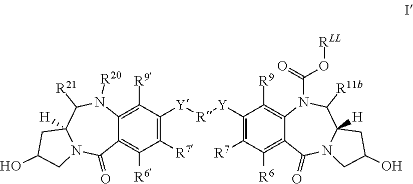
wherein R⁶, R⁷, R⁹, R^{11b}, Y, R″, Y′, R^{6′}, R⁷, R^{9′}, R²⁰ and R²¹, are as defined in claim 1;
R^{LL} is a linker for connection to a cell binding agent, which is selected from:
(iiia): 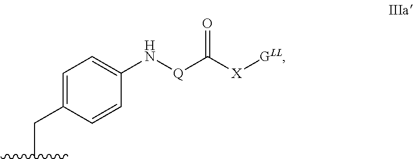
where Q and X are as defined in claim 1 and G^{LL} is a linker connected to an antibody or an active fragment thereof; and 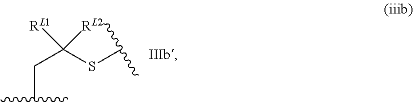
where R^{L1} and R^{L2} are as defined in claim 1;
wherein p is an integer of from 1 to 20.

18. A conjugate according to claim 17, wherein C^{LL} is selected from:

| | |
|---|---|
| 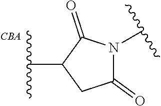 [SMILES: *C1CC(=O)N(*)C1=O] | (G^{LL1-1}) |
| 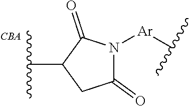 | (G^{LL1-2}) |
| 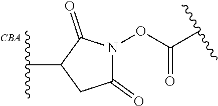 [SMILES: *C(=O)ON1C(=O)CC(*)C1=O] | (G^{LL2}) |
| 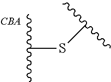 [SMILES: *S*] | (G^{LL3-1}) |
| 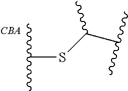 [SMILES: *SC(*)C] | (G^{LL3-2}) |
| 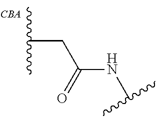 [SMILES: *CC(=O)N*] | (G^{LL4}) |
| 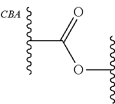 [SMILES: *OC(*)=O] | (G^{LL5}) |
| 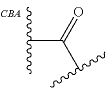 [SMILES: *C(*)=O] | (G^{LL6}) |
| 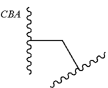 [SMILES: *C*] | (G^{LL7}) |
| 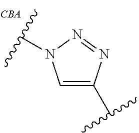 [SMILES: *c1cn(*)nn1] | (G^{LL8-1}) |
| 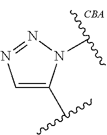 [SMILES: *c1cnnn1*] | (G^{LL8-2}) |
| 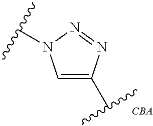 [SMILES: *c1cn(*)nn1] | (G^{LL9-1}) |
| 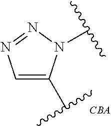 [SMILES: *c1cnnn1*] | (G^{LL9-2}) |

where Ar represents a C₅₋₆ arylene group.

19. A conjugate according to claim 17, wherein D^{L} is of formula (Id′): 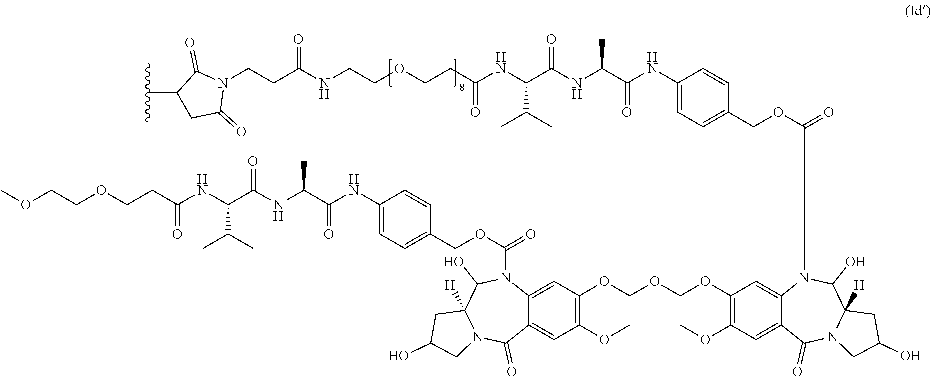
where Q is selected from:
(a) —CH₂—;
(b) —C₃H₆—; and

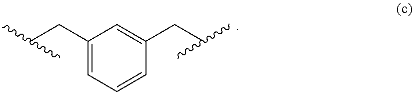 [SMILES: *Cc1cccc(C*)c1]

20. A compound of Formula IV: 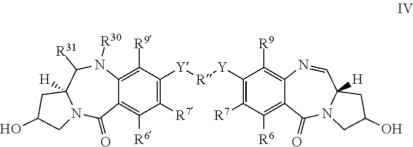
wherein R⁶, R⁷, R⁹, Y, R″, Y′, R^{6′}, R⁷ and R^{9′}, are as defined in claim 1; either:
(a) R³⁰ is H and R³¹ is H;
(b) R³⁰ is H and R³¹ is ═O; or
(c) R³⁰ and R³¹ form a double bond between the N and C atoms to which they are attached.

21. A composition comprising a mixture of conjugates according to claim 17, wherein the average p in the mixture of conjugate compounds is about 1 to about 8.

22. (canceled)

23. A pharmaceutical composition comprising the conjugate of claim 17, and a pharmaceutically acceptable diluent, carrier or excipient.

24.-25. (canceled)

26. A method of treating a proliferative disease comprising administering a therapeutically effective amount of a conjugate according to claim 17.
